SEQUENCE LISTING (31 sequences)

SEQ ID NO: 1 - DNA, 33 bp, Artificial Sequence
  A synthetic oligonucleotide
gggttattggagacggtaccgtctcctcccccc

SEQ ID NO: 2 - DNA, 33 bp, Artificial Sequence
  A synthetic oligonucleotide
ggggggaggagacggtaccgtctccaataaccc

SEQ ID NO: 3 - DNA, 18 bp, Artificial Sequence
  A synthetic oligonucleotide
cgtctcntattagtagaa

SEQ ID NO: 4 - DNA, 17 bp, Artificial Sequence
  A synthetic oligonucleotide
ttttgctcccngagacg

SEQ ID NO: 5 - DNA, 18 bp, Artificial Sequence
  A synthetic oligonucleotide
ttctactaatangagacg

SEQ ID NO: 6 - DNA, 17 bp, Artificial Sequence
  A synthetic oligonucleotide
cgtctcngggagcaaaa

SEQ ID NO: 7 - DNA, 11 bp, Artificial Sequence
  A synthetic oligonucleotide
tattagtagaa

SEQ ID NO: 8 - DNA, 10 bp, Artificial Sequence
  A synthetic oligonucleotide
gggagcaaaa

SEQ ID NO: 9 - DNA, 15 bp, Artificial Sequence
  A synthetic oligonucleotide
gggttattagtagaa

SEQ ID NO: 10 - DNA, 13 bp, Artificial Sequence
  A synthetic oligonucleotide
ttttgctcccccc

SEQ ID NO: 11 - DNA, 15 bp, Artificial Sequence
  A synthetic oligonucleotide
ttctactaataaccc

SEQ ID NO: 12 - DNA, 13 bp, Artificial Sequence
  A synthetic oligonucleotide
ggggggagcaaaa

SEQ ID NO: 13 - DNA, 80 bp, Artificial Sequence
  A synthetic oligonucleotide
cacacacgtctcgtattagtagaaacaaggtcgtttttaaactattcgacactaattgatggccatccgaattcttttgg

SEQ ID NO: 14 - DNA, 67 bp, Artificial Sequence
  A synthetic oligonucleotide
cacacacgtctccgggagcgaaagcaggtcaattatattcaatatggaaagaataaaagaactaagg

SEQ ID NO: 15 - DNA, 89 bp, Artificial Sequence
  A synthetic oligonucleotide
cacacacgtctcgtattagtagaaacaaggcattttttcatgaaggacaagctaaattcactatttttgccgtctgagctcttcaatgg

SEQ ID NO: 16 - DNA, 67 bp, Artificial Sequence
  A synthetic oligonucleotide
cacacacgtctccgggagcgaaagcaggcaaaccatttgaatggatgtcaatccgactttacttttc

SEQ ID NO: 17 - DNA, 103 bp, Artificial Sequence
  A synthetic oligonucleotide
ccaacccgtctcctattagtagaaacaaggtacttttttggacagtatggatagcaaatagtagcattgccacaactatctcaatgcatgtgtgaggaaggag

SEQ ID NO: 18 - DNA, 67 bp, Artificial Sequence
  A synthetic oligonucleotide
ccaacccgtctccgggagcgaaagcaggtactgattcaaaatggaagattttgtgcgacaatgcttc

SEQ ID NO: 19 - DNA, 40 bp, Artificial Sequence
  A synthetic oligonucleotide
cacacacgtctcctattagtagaaacaagggtgtttttcc

SEQ ID NO: 20 - DNA, 45 bp, Artificial Sequence
  A synthetic oligonucleotide
cacacacgtctccgggagcaaaagcaggggaaaataaaaacaacc

SEQ ID NO: 21 - DNA, 47 bp, Artificial Sequence
  A synthetic oligonucleotide
cacacacgtctcctattagtagaaacaagggtatttttctttaattg

SEQ ID NO: 22 - DNA, 42 bp, Artificial Sequence
  A synthetic oligonucleotide
cacacacgtctccgggagcaaaagcagggtagataatcactc

SEQ ID NO: 23 - DNA, 46 bp, Artificial Sequence
  A synthetic oligonucleotide
cacacacgtctcctattagtagaaacaaggagttttttgaacaaac

SEQ ID NO: 24 - DNA, 48 bp, Artificial Sequence
  A synthetic oligonucleotide
cacacacgtctccgggagcgaaagcaggagtttaaatgaatccaaacc

SEQ ID NO: 25 - DNA, 47 bp, Artificial Sequence
  A synthetic oligonucleotide
cacacacgtctcctattagtagaaacaaggtagttttttactccagc

SEQ ID NO: 26 - DNA, 41 bp, Artificial Sequence
  A synthetic oligonucleotide
cacacacgtctccgggagcaaaagcaggtagatattgaaag

SEQ ID NO: 27 - DNA, 53 bp, Artificial Sequence
  A synthetic oligonucleotide
cacacacgtctcctattagtagaaacaagggtgttttttattattaaataagc

SEQ ID NO: 28 - DNA, 46 bp, Artificial Sequence
  A synthetic oligonucleotide
cacacacgtctccgggagcaaaagcagggtgacaaagacataatgg

SEQ ID NO: 29 - RNA, 95 nt, Artificial Sequence
  A synthetic nucleic acid
gggcgaaagcccagaagguccugaugaggccgaaaggccgaaacuccaguccucuacgaaaguagagcugaugaagcgaaagcucgaaaguggag

SEQ ID NO: 30 - RNA, 115 nt, Artificial Sequence
  A synthetic nucleic acid
gggcgaaagcccagaagguccugaugaggccgaaaggccgaaacuccaguggcaguccuguugaaaaacagagaagccaaccagagaaacacacguugugguauauuaccuggua

SEQ ID NO: 31 - RNA, 110 nt, Artificial Sequence
  A synthetic nucleic acid
gggcgaaagcccagaagguccugaugaggccgaaaggccgaaacuccaaagggucggcauggcaucuccaccuccucgcgguccgaccugggcuacuucgguaggcuaag

(19) United States
(12) Patent Application Publication
(10) Pub. No.: US 2009/0017444 A1
(43) Pub. Date: Jan. 15, 2009

(54) Screening method for modulators of viral transcription or replication

(75) Inventors: Yoshihiro Kawaoka, Middleton, WI (US); Gabriele Neumann, Madison, WI (US)

Correspondence Address: SCHWEGMAN, LUNDBERG & WOESSNER, P.A., P.O. BOX 2938, MINNEAPOLIS, MN, 55402, US

(73) Assignee: WISCONSIN ALUMNI RESEARCH FOUNDATION, MADISON, WI (US)

(21) Appl. No.: 11/810,956

(22) Filed: Jun. 7, 2007

Related U.S. Application Data
(60) Provisional application No. 60/804,341, filed on Jun. 9, 2006.

(51) Int. Cl. C12Q 1/70

(52) U.S. Cl. 435/5

(57) ABSTRACT

The invention provides methods to identify modulators of viral transcription or replication.

### CROSS-REFERENCE TO RELATED APPLICATIONS

This application claims the benefit of the filing date of U.S. application Ser. No. 60/804,341, filed Jun. 9, 2006, the disclosure of which is incorporated by reference herein.

### BACKGROUND

The ability to generate infectious RNA viruses from cloned cDNAs has contributed greatly to the biological understanding of these pathogens and hence to improved methods of disease control (Palese et al., 1996). However, this progress had been relatively limited for negative-sense as compared with positive-sense RNA viruses, because neither the genomic viral RNA (vRNA) nor the antigenomic complementary RNA (cRNA) of negative-sense RNA viruses can serve as a direct template for protein synthesis. Rather, the vRNA, after its encapsidation by viral nucleoprotein (NP), must be transcribed into positive-sense mRNA by the viral RNA polymerase complex. Thus, the minimal replication unit is formed by the genomic vRNA complexed with NP and the polymerase proteins. Despite these obstacles, reverse genetics methods have been established to produce nonsegmented negative-sense RNA viruses, including rabies virus (Schnell et al., 1994), vesicular stomatitis virus (Lawson et al., 1995; Whelan et al., 1995), measles virus (Radecke et al., 1995), respiratory syncytial virus (Collins et al., 1995), Sendai virus (Garcin et al., 1995; Kato et al., 1996), rinderpest virus (Baron et al., 1997), human parainfluenza virus type 3 (Hoffman et al., 1997) and SV5 (He et al., 1997).

The Orthomyxoviridae, Arenaviridae, and Bunyaviridae families contain segmented, negative strand RNA genomes and include several human and animal pathogens, for example, influenza virus types A, B, and C (Orthomyxoviridae), lymphocytic choriomeningitis virus (LCMV) (Arenaviridae), and encephalitic and hemorrhagic fever viruses (Bunyaviridae, Arenaviridae). Their genomes consist of two (Arenaviridae), three (Bunyaviridae), or six to eight (Orthomyxoviridae) single-stranded RNA molecules of negative polarity (complementary to mRNA). The vRNAs interact with NP and viral RNA-dependent RNA-polymerase to form ribonucleoprotein complexes (RNPs). The RNPs are surrounded by a lipid bilayer derived from the host cell. Inserted in this envelope are viral glycoproteins, which are essential for receptor binding and entry into the host cell. Thus, generating segmented negative-sense RNA viruses from cloned cDNAs poses a formidable challenge, as one must produce a separate vRNA for each gene segment.

Bridgen and Elliott (1996) produced a Bunyamwera virus (family Bunyaviridae) from cloned cDNAs encoding three segments of antigenomic, positive-sense vRNA. However, the efficiency of virus recovery was low. None of the orthomyxoviruses, which contain six (thogotovirus), seven (influenza C virus) or eight (influenza A and B viruses) segments of negative-sense RNA have been produced entirely from cloned cDNAs. This lag in progress has been felt most acutely in efforts to control influenza virus infections.

Palese and colleagues (Enami et al., 1990) pioneered the reverse genetics, helper virus-dependent system for influenza A virus (FIG. 1A). In their approach, RNP complexes are generated by in vitro vRNA synthesis in the presence of purified polymerase and NP proteins, and then used to transfect eukaryotic cells. Subsequent infection with influenza A helper virus results in the generation of viruses possessing a gene derived from cloned cDNA. A second method, developed by Neumann et al. (1994), is based on the in vivo synthesis of vRNA by RNA polymerase I (FIG. 1B), a cellular enzyme that transcribes ribosomal RNA that lacks both a 5′ cap and a 3′ polyA tail. Cells infected with influenza virus and transfected with a plasmid containing cloned influenza virus cDNA, flanked by murine RNA polymerase I promoter and terminator sequences, led to the production of transfectant viruses. With both methods, however, transfectants must be selected from a vast background of helper viruses, which requires a strong selection system and complicates the generation of growth-defective viruses.

A system to generate replication-incompetent virus-like particles (VLPs) was developed by Mena et al. (1996), in which an influenza virus-like vRNA encoding a reporter gene is transcribed in vitro and transfected into eukaryotic cells. All ten influenza virus proteins are expressed from plasmids under the control of a T7 RNA polymerase promoter. When the transfected cells are infected with a recombinant vaccinia virus that expressed T7 RNA polymerase, they produced influenza VLPs. However, the efficiency of the system is low: in 25% of the experiments, the investigators failed to detect reporter gene expression. Moreover, vaccinia virus expresses more than 80 proteins, any of which could affect the influenza viral life cycle. More recently, reverse genetics was used to produce influenza virus entirely from cloned DNA (Neumann et al., 2000).

Thus, what is needed is a method to prepare segmented, negative strand RNA viruses, e.g., orthomyxoviruses such as influenza A viruses, entirely from cloned cDNAs.

### SUMMARY OF THE INVENTION

The invention provides a plurality of isolated vectors useful to screen for drugs or host factors that modulate viral RNA transcription and/or replication. The vectors include vectors having a promoter linked to sequences for a viral polymerase, e.g., to each viral protein which forms a viral polymerase, a vector having a promoter linked to sequences for a reporter flanked by viral sequences for packaging, transcription and/or replication, and optionally a vector having a promoter linked to sequences for a viral capsid or nucleoprotein which forms a complex with viral RNA.

In one embodiment, the reporter sequences are linked to viral sequences which include 5′ orthomyxovirus non-coding sequences and include 3′ orthomyxovirus non-coding sequences. Exemplary reporter sequences include but are not limited to fluorescent protein, e.g., green fluorescent protein, sequences luciferase sequences, beta-galactosidase sequences, beta-glucuronidase sequences, or immunogenic protein sequences, e.g., those coding from a protein which binds an antibody or other ligand specific for the protein, or others that are detectable, e.g., optically detectable. The promoter which is operably linked to reporter sequences, which may be in antisense (antigenomic orientation for negative-sense viruses), may be, for example, a RNA polymerase I promoter, a RNA polymerase II promoter, a RNA polymerase III promoter, a T7 promoter, or a T3 promoter. The transcription termination sequence may be a RNA polymerase I transcription termination sequence, a RNA polymerase II transcription termination sequence, a RNA polymerase III transcription termination sequence, or a ribozyme.

In one embodiment, the invention provides the following isolated vectors: a vector for protein expression comprising a promoter operably linked to an influenza virus PA cDNA linked to a transcription termination sequence, a vector for protein expression comprising a promoter operably linked to an influenza virus PB1 cDNA linked to a transcription termination sequence, a vector comprising a promoter operably linked to an influenza virus PB2 cDNA linked to a transcription termination sequence, a vector for protein expression comprising a promoter operably linked to an influenza virus NP cDNA linked to a transcription termination sequence, and a vector for vRNA comprising a promoter operably linked to reporter sequences which are flanked by influenza virus sequences for packaging, replication and/or transcription, linked to a transcription termination sequence, and a composition or host cell comprising those vectors. Thus, the invention provides isolated vectors or plasmids, which express or encode viral proteins and express or encode vRNA, e.g., recombinant vRNA. In one embodiment, the vectors comprise influenza cDNA, e.g., influenza A (e.g., any influenza A gene including any of the 15 HA or 9 NA subtypes), B or C DNA (see Chapters 45 and 46 of Fields Virology (Fields et al. (eds.), Lippincott-Raven Publ., Philadelphia, Pa. (1996), which are specifically incorporated by reference herein), although it is envisioned that the gene(s) of any virus may be employed in the vectors or methods of the invention. For instance, the same approach may be employed for other viruses such as nonsegmented negative strand RNA viruses (i.e., Paramyxoviridae, Rhabdoviridae, and Filoviridae), or other segmented negative strand RNA viruses, e.g., Arenaviridae and Bunyaviridae.

In one embodiment, the reporter sequences are flanked by influenza virus PA cDNA sequences, e.g., at least 5′ and 3′ noncoding PA sequences. In another embodiment, the reporter sequences are flanked by influenza virus PB1 cDNA sequences, e.g., at least 5′ and 3′ noncoding PB1 sequences. In yet another embodiment, the reporter sequences are flanked by influenza virus PB2 cDNA sequences, e.g., at least 5′ and 3′ noncoding PB2 sequences. In a further embodiment, the reporter sequences are flanked by influenza virus HA cDNA sequences, e.g., at least 5′ and 3′ noncoding HA sequences. In yet a further embodiment, the reporter sequences are flanked by influenza virus NP cDNA sequences, e.g., at least 5′ and 3′ noncoding NP sequences. In one embodiment, the reporter sequences are flanked by influenza virus NA cDNA sequence, e.g., at least 5′ and 3′ noncoding NA sequences. In another embodiment, the reporter sequences are flanked by influenza virus M cDNA sequences, e.g., at least 5′ and 3′ noncoding M sequences. In a further embodiment, the reporter sequences are flanked by influenza virus NS cDNA sequences, e.g., at least 5′ and 3′ noncoding NS sequences. The cDNA may be in the sense or antisense orientation relative to the promoter.

Any promoter may be employed to express a viral protein. A promoter for the vectors includes but is not limited to a RNA polymerase I promoter, a RNA polymerase II promoter, a RNA polymerase III promoter, a T7 promoter, and a T3 promoter. Each vector comprising an open reading frame may include a transcription termination sequence such as a RNA polymerase I transcription termination sequence, a RNA polymerase II transcription termination sequence, a RNA polymerase III transcription termination sequence, or a ribozyme. Preferred promoters for the vectors for vRNA include, but are not limited to, a RNA polymerase I promoter, a RNA polymerase II promoter, a RNA polymerase III promoter, a T7 promoter, and a T3 promoter. In one embodiment, the vector or plasmid which expresses vRNA comprises a promoter, e.g., a RNA polymerase I, suitable for expression in a particular host cell, e.g., avian or mammalian host cells such as canine, feline, equine, bovine, ovine, or primate cells including human cells. In one embodiment, the RNA polymerase I promoter is a human RNA polymerase I promoter. The vectors or plasmids comprising DNA useful to prepare influenza vRNA may comprise RNA polymerase I transcription termination sequences. Preferred transcription termination sequences for the vectors for vRNA include, but are not limited to, a RNA polymerase I transcription termination sequence, a RNA polymerase II transcription termination sequence, or a RNA polymerase III transcription termination sequence, or a ribozyme.

A plurality of the vectors of the invention may be physically linked or each vector may be present on an individual plasmid or other, e.g., linear, nucleic acid delivery vehicle.

Also provided is a method which includes contacting a host cell with a plurality of the vectors of the invention, e.g., sequentially or simultaneously, for example, employing a composition of the invention having one or more vectors. In one embodiment, the host cell is contacted with a plurality of vectors with viral sequences which together do not generate infectious virus and a vector with heterologous sequences for a reporter. In one embodiment, the host cell is contacted with a plurality of vectors which together generate infectious virus and a vector with heterologous sequences for a reporter. In one embodiment, the host cell is contacted with a plurality of vectors, one of which has sequences for a heterologous glycoprotein, others that include sequences for at least a viral polymerase, and another with heterologous sequences for a reporter. For example, a host cell is contacted with a vector having sequences for a non-influenza virus glycoprotein, vectors for influenza virus polymerase proteins and optionally a vector for influenza virus nucleoprotein, and a vector with heterologous reporter sequences.

The invention also provides a method to identify modulators of a viral polymerase, e.g., a viral RNA polymerase. The method includes providing a host cell having the vectors of the invention; contacting the host cell and at least one agent, and determining whether the one or more agents modulate the amount or activity of the one or more of the viral polymerase proteins. In one embodiment, the host cell is contacted with a library of agents. For instance, the host cell may be contacted with a cDNA library or siRNA library.

The invention also provides vectors and methods for positive strand viruses, e.g., positive-sense RNA viruses. Thus, the invention provides a vector comprising a DNA segment comprising RNA polymerase I transcription initiation sequences operably linked to a second DNA segment comprising sequences from a positive-sense RNA virus, optionally operably linked to a third DNA segment comprising RNA polymerase I transcription termination sequences. RNA polymerase I transcription is highly efficient and has high fidelity. For positive-sense RNA viruses whose genomic RNA is uncapped (e.g., pestiviruses; hepatitis C virus; and Picornaviridae, including poliovirus, rhinoviruses, hepatitis A virus, and foot and mouth disease virus), a cDNA encoding the full-length genome is introduced in genomic-sense orientation between RNA polymerase I promoter and terminator sequences. Transfection of the resulting plasmid into permissive host cells yields genomic RNA for replication. A number of positive-sense RNA viruses contain capped genomic RNAs (e.g., flaviviruses, including dengue fever virus and several encephalitis viruses). While RNA polymerase I transcripts are not capped, a cDNA encoding the full-length genome of RNA viruses having capped genomic RNAs is introduced in antigenomic-sense orientation in a RNA polymerase I transcription vector. Following transfection of the resulting plasmid, cellular RNA polymerase I transcribes an antigenomic (uncapped) RNA. Moreover, cotransfection with protein expression plasmids for the proteins required for replication results in the replication of the antigenomic RNA, hence yielding genomic RNA.

### BRIEF DESCRIPTION OF THE FIGURES

FIG. 1. Schematic diagram of established reverse genetics systems. In the RNP transfection method (A), purified NP and polymerase proteins are assembled into RNPs with use of in vitro-synthesized vRNA. Cells are transfected with RNPs, followed by helper virus infection. In the RNA polymerase I method (B), a plasmid containing the RNA polymerase I promoter, a cDNA encoding the vRNA to be rescued, and the RNA polymerase I terminator is transfected into cells. Intracellular transcription by RNA polymerase I yields synthetic vRNA, which is packaged into progeny virus particles upon infection with helper virus. With both methods, transfectant viruses (i.e., those containing RNA derived from cloned cDNA), are selected from the helper virus population.

FIG. 2. Schematic diagram of the generation of RNA polymerase I constructs. cDNAs derived from influenza virus were amplified by PCR, digested with BsmBI and cloned into the BsmBI sites of the pHH21 vector (E. Hoffmann, Ph.D. thesis, Justus, Liebig-University, Giessen, Germany), which contains the human RNA polymerase I promoter (P) and the mouse RNA polymerase I terminator (T). The thymidine nucleotide upstream of the terminator sequence (*T) represents the 3′ end of the influenza viral RNA. Influenza A virus sequences are shown in bold face letters.

FIG. 3. Proposed reverse genetics method for generating segmented negative-sense RNA viruses. Plasmids containing the RNA polymerase I promoter a cDNA for each of the eight viral RNA segments, and the RNA polymerase I terminator are transfected into cells together with protein expression plasmids. Although infectious viruses can be generated with plasmids expressing PA, PB1, PB2, and NP, expression of all remaining structural proteins (shown in brackets) increases the efficiency of virus production depending on the virus generated.

FIG. 4. Detection of the FLAG epitope in cells infected with a transfectant virus. Antibody staining was used to identify the NA in MDCK cells infected with either PR8-WSN-FL79 (A, D) or A/WSN/33 wild-type virus (B, E), or on mock-infected MDCK cells (C, F). Nine hours after infection, cells were fixed with paraformaldehyde, treated with Triton X-100 and incubated with either anti-FLAG (A-C) or anti-WSN NA (D-F) monoclonal antibodies. Intensive Golgi staining (red) is apparent in positive samples (A, D, and E).

FIG. 5. Recovery of PA mutants. The PA gene of each virus was amplified by RT-PCR with primers that yield a 1226 bp fragment (position 677 to 1903 of the mRNA, lanes 1, 3, 5), which was then digested with the restriction enzyme Bsp120I (at position 846 of the mRNA, lanes 4, 7) or PvuII (at position 1284 of the mRNA, lanes 2, 6). The presence of Bsp120I or PvuII sites in the PCR products yielded either 169 bp and 1057 bp or 607 bp and 619 bp fragments, respectively. MW=molecular weight markers.

FIGS. 6A and 6B. Primers employed to amplify influenza sequences.

FIG. 7. Schematic diagram of VLP generation strategy. The plasmid contains the GFP gene (derived from pEGFP-N1; Clontech, Palo Alto, Calif.) in antisense orientation between the 5′ and 3′ noncoding regions of influenza A virus segment 5, flanked by the human RNA polymerase I promoter and the mouse RNA polymerase I terminator. Individual protein expression plasmids and a plasmid containing the RNA polymerase I promoter, a cDNA encoding the GFP reporter gene, and the RNA polymerase I terminator are transfected into 293T cells. Intracellular transcription by RNA polymerase I yields GFP vRNA of negative polarity, as indicated by inverted letters. Supernatants containing VLPs are harvested, mixed with influenza helper virus and inoculated into MDCK cells.

FIG. 8. Plasmid-based system having 5 plasmids with influenza virus sequences to screen modulators of viral replication. 4 of the plasmids have an influenza cDNAs for one of PA, PB1, PB2 and NP, to express viral proteins, and 1 of the plasmids has influenza viral sequences, e.g., from the NP cDNA, flanking a reporter, e.g., a sequence encoding luciferase, for packing and expressing vRNA.

FIG. 9. Plasmid-based system having 3 plasmids with influenza virus sequences to screen modulators of viral replication. 1 of the plasmids has a cDNA for three viral proteins, i.e., PA, PB1 and PB2, 1 of the plasmids has a cDNA for NP, and the third plasmid has influenza sequences flanking reporter sequences.

FIG. 10. Exemplary protocol for testing compounds using the plasmid based system, a luciferase reporter, and a T7 RNA polymerase system.

FIG. 11. Vector map of pCAGGS-2Ribo-1. In vertebrate cells, RNA polymerase II drives RNA transcription of the double ribozyme cassette, which is under the control of the strong chicken β-actin promoter/CMV enhancer cassette. Viral cDNA is cloned downstream of the hammerhead ribozyme site (HamRz) and upstream of the HDV ribozyme site (HDVRz).

FIG. 12. Exemplary hammerhead (HH), hairpin (HP) and HDV ribozyme sequences. Solid arrows indicate self-processing sites.

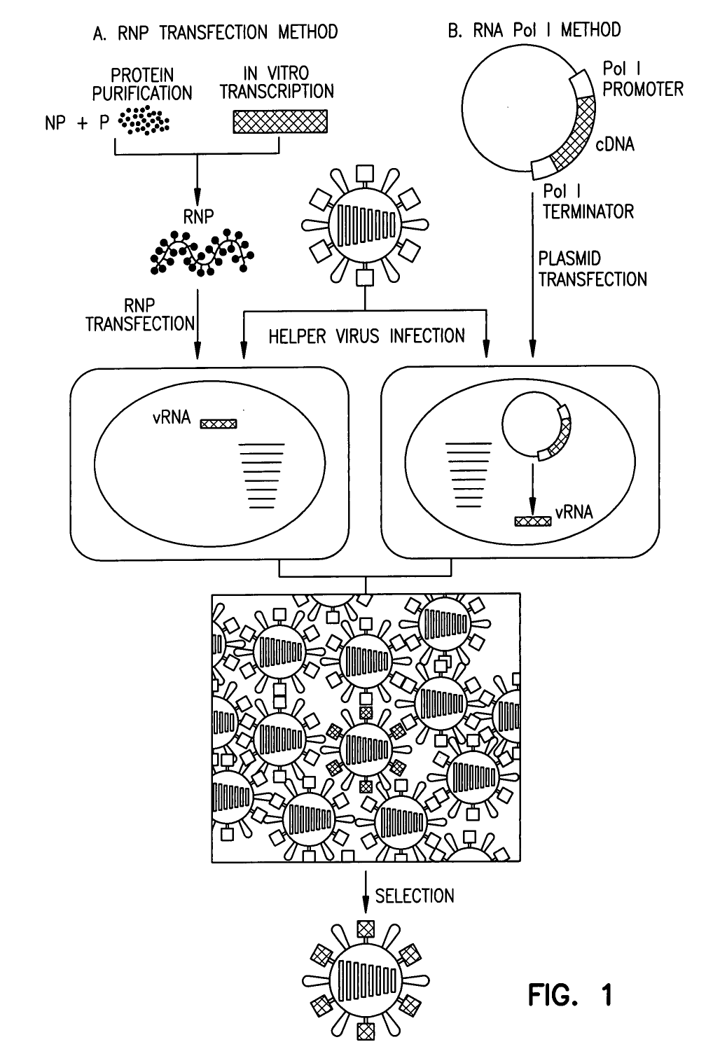

### DETAILED DESCRIPTION OF THE INVENTION

### Definitions

As used herein, the terms “isolated and/or purified” refer to in vitro preparation, isolation and/or purification of a vector or plasmid of the invention, so that it is not associated with in vivo substances, or is substantially purified from in vitro substances. As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

### Influenza Virus Replication

Influenza A viruses possess a genome of eight single-stranded negative-sense viral RNAs (vRNAs) that encode a total of ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cDNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, influenza B virus does not have a M2 protein with ion channel activity. Similarly, influenza C virus does not have a M2 protein with ion channel activity.

Generally, incorporation sequences are present in the about 150 to about 250 nucleotides at one or each end of the coding region for each influenza vRNA segment. In one embodiment, influenza virus incorporation sequences comprise sequences corresponding to the 3′ end of NA vRNA including sequences corresponding to the N-terminus of the NA coding region, e.g., 37 nucleotides of the 3′ end of type A NA vRNA including 19 nucleotides of 3′ noncoding sequence and at least nucleotides corresponding to the first 19 coding nucleotides for NA and, optionally, incorporation sequences corresponding to the 5′ end of NA vRNA including sequences corresponding to the C-terminus of the NA coding region, e.g., 67 nucleotides of the 5′ end of type A NA vRNA including 28 nucleotides of 5′ noncoding sequence and at least 39 nucleotides corresponding to the 39 3′ coding nucleotides of NA. In another embodiment, influenza virus incorporation sequences comprise sequences corresponding to the 3′ end of NS vRNA including sequences corresponding to the N-terminus of the NS coding region. In yet another embodiment, influenza virus incorporation sequences comprise sequences corresponding to the 5′ end of HA vRNA including sequences corresponding to the C-terminus of the HA coding region, e.g., 135 nucleotides of the 5′ end of type A HA vRNA including 45 nucleotides of 5′ noncoding sequence and at least 80 nucleotides corresponding to the 80 3′ coding nucleotides of HA and, optionally, incorporation sequences corresponding to the 3′ end of HA vRNA, including sequences corresponding to the N-terminus of the HA coding region, e.g., 36 nucleotides of the 3′ end of type A HA vRNA including 33 nucleotides of 3′ noncoding sequence and at least 3 nucleotides corresponding to the first 3 coding nucleotides of HA. In a further embodiment, influenza virus incorporation sequences comprise sequences corresponding to the 5′ end of PB2 vRNA including sequences corresponding to the C-terminus of the PB2 coding region. In another embodiment, influenza virus incorporation sequences comprise sequences corresponding to the 3′ end of M vRNA including sequences corresponding to the N-terminus of the M coding region, e.g., 247 nucleotides of the 3′ end of type A M vRNA including 26 nucleotides of 3′ noncoding sequence and 221 nucleotides of sequence corresponding to the N-terminus of the M coding region, and sequences corresponding to the 5′ end of M vRNA including incorporation sequences corresponding to the C-terminus of the M coding region, e.g., 242 nucleotides of the 5′ end of type A M vRNA including 23 nucleotides of 3′ noncoding sequence and 219 nucleotides of sequence corresponding to the last 219 nucleotide for the C-terminus of the M coding region. In another embodiment, influenza virus incorporation sequences comprise sequences corresponding to the 5′ end of NS vRNA including sequences corresponding to the N-terminus of the NS coding region, e.g., sequences including the 3′ noncoding sequence and at least the first 30 nucleotides corresponding to the N-terminus of the NS coding region, and sequences corresponding to the 5′ end of NS vRNA including incorporation sequences corresponding to the C-terminus of the NS coding region, e.g., sequences including the 5′ noncoding sequence and at least the last 30 nucleotides of sequence corresponding to the C-terminus of the NS coding region. In one embodiment, influenza virus incorporation sequences comprise sequences corresponding to the 5′ end of PB1 vRNA including sequences corresponding to the N-terminus of the PB1 coding region and sequences corresponding to the 5′ end of PB1 vRNA including incorporation sequences corresponding to the C-terminus of the PB1 coding region. In yet another embodiment, influenza virus incorporation sequences comprise sequences corresponding to the 5′ end of PA vRNA including sequences corresponding to the N-terminus of the PA coding region and sequences corresponding to the 5′ end of PA vRNA including incorporation sequences corresponding to the C-terminus of the PA coding region. Influenza virus “incorporation sequences,” as used herein, are sequences which, when present in vRNA with corresponding (homologous) 3′ and 5′ noncoding regions, result in the incorporation of a nucleic acid molecule comprising those sequences into virions and the maintenance of that molecule in virions during repeated passage.

In another embodiment, the 3′ NA incorporation sequences correspond to nucleotides 1 to 183, nucleotides 1 to 90, nucleotides 1 to 45, nucleotides 1 to 21, nucleotides 1 to 19 or any integer between 19 and 183, of the N-terminal NA coding region, and may include a mutation at the NA initiation codon. In another embodiment, the 5′ NA incorporation sequences correspond to sequences in the C-terminal coding region of NA, sequences corresponding to the 3′ most 39, 78, or 157, or any integer between 1 and 157, nucleotides for C-terminal NA coding region. In another embodiment, the S′ HA incorporation sequences correspond to sequences in the C-terminal coding region of HA, sequences corresponding to the 3′ most 75, 80, 268, 291, or 518, or any integer between 1 and 518, nucleotides of the C-terminal HA coding region. The 3′ HA incorporation sequences correspond to nucleotides 1 to 3, 1 to 6, 1 to 9, 1 to 15, 1 to 216, 1 to 468, or any integer between 1 and 468, of the N-terminal HA coding region. In one embodiment, the 3′ PB1 incorporation sequences correspond to nucleotides 1 to 250, nucleotides 1 to 200, nucleotides 1 to 150, or any integer between 1 and 250, of the N-terminal PB1 coding region. In one embodiment, the 5′ PB1 incorporation sequences correspond to the 3′ most nucleotides, e.g., the 3′ 1 to 250 nucleotides, 1 to 200 nucleotides, nucleotides 1 to 150, or any integer between 1 and 250, of the C-terminal PB1 coding region. In one embodiment, the 3′ PA incorporation sequences correspond to nucleotides 1 to 250, nucleotides 1 to 200, nucleotides 1 to 150, or any integer between 1 and 250, of the N-terminal PA coding region. In one embodiment, the 5′ PA incorporation sequences correspond to the 3′ most nucleotides, e.g., the 3′ 1 to 250 nucleotides, 1 to 200 nucleotides, nucleotides 1 to 150, or any integer between 1 and 250, of the C-terminal PA coding region. In another embodiment, the 3′ M incorporation sequences correspond to nucleotides 1 to 250, nucleotides 1 to 242, nucleotides 1 to 240, or any integer between 1 and 250, of the N-terminal M coding region, and may include a mutation at the M initiation codon. In another embodiment, the 5′ M incorporation sequences correspond to sequences in the C-terminal coding region of M, sequences corresponding to the 3′ most 50, 100, or 220, or any integer between 1 and 250, nucleotides for C-terminal M coding region. In another embodiment, the 3′ NS incorporation sequences correspond to nucleotides 1 to 250, nucleotides 1 to 200, nucleotides 1 to 150, nucleotides 1 to 30, nucleotides 1 to 20 or any integer between 1 and 250, of the N-terminal NS coding region, and may include a mutation at the NS initiation codon. In another embodiment, the 5′ NS incorporation sequences correspond to sequences in the C-terminal coding region of NS, sequences corresponding to the 3′ most 10, 30, 150, 200 or 250, or any integer between 1 and 250, nucleotides for the C-terminal NS coding region.

### Thogotovirus

Thogotoviruses (THOV) represent a new genus in the family of Orthomyxoviridae. They are transmitted by ticks and have been found in domestic animals, including camels, goats, and cattle. Consequently, THOV can replicate in tick and vertebrate cells. The THOV genome comprises six segments of single-stranded, negative-sense RNA. The proteins encoded by the three largest segments show significant homology to the influenza virus polymerase proteins PB2, PB1, and PA. Segment 5 encodes a protein related to influenza virus NP. The THOV glycoprotein, which is encoded by segment 4, is not homologous to either influenza virus HA or NA, but it shows sequence similarity to the Baculovirus glycoprotein. The smallest segment is thought to encode a matrix protein and does not resemble any of the influenza virus proteins. Like influenza virus, both the 3′ and 5′ ends of the vRNA are required for promoter activity, and this activity is located in the terminal 14 and 15 nucleotides of the 3′ and 5′ ends of the vRNA, respectively.

The mRNA synthesis of THOV is primed by host cell-derived cap structures. However, in contrast to influenza virus, only the cap structures (without additional nucleotides) are cleaved from cellular mRNAs (Albo et al., 1996; Leahy et al., 1997; Weber et al., 1996). In vitro cleavage assays revealed that both the 5′ and 3′ ends of vRNA are required for endonuclease activity (Leahy et al., 1998), but addition of a model cRNA promoter does not stimulate endonuclease activity (Leahy et al., 1998), as has been shown for influenza virus (Hagen et al., 1994). A ‘hook’ structure has been proposed for THOV (Leahy et al., 1997; Weber et al., 1997), which is similar to the corkscrew structure proposed for influenza virus. This ‘hook’ structure, however, is only found in the THOV vRNA promoter. The cRNA promoter sequence does not allow the formation of base pairs between positions 2 and 9, and between 3 and 8 at the 5′ end of the cRNA. Alterations at positions 3 or 8 to allow base-pairing between these nucleotides stimulates endonuclease activity, which is strong supporting evidence of the proposed ‘hook’ structure (Leahy et al., 1998). Moreover, this structure might be crucial for the regulation of the THOV life cycle; the vRNA promoter, forming the ‘hook’ structure, may stimulate PB2 endonuclease activity, thereby allowing transcription. The cRNA promoter, in contrast, may not form the ‘hook’ structure and may therefore be unable to stimulate endonuclease activity, thus resulting in replication.

### Bunyaviridae

The family Bunyaviridae includes several viruses that cause hemorrhagic or encephalitic fevers in humans (e.g., Rift fever valley, Hantaan, La Crosse, and Crimean-Congo hemorrhagic fever). The spherical and enveloped virions contain three segments of single-stranded, negative-sense RNA (reviewed in Elliott, 1997). The largest segment (L) encodes the viral RNA polymerase protein (L protein), whereas the M segment encodes the two viral glycoproteins G1 and G2, and a nonstructural protein (NSm). The smallest segment (S) encodes the nucleocapsid protein (N) and a second nonstructural protein (NSs). Virus replication and transcription take place in the cytoplasm, and newly assembled virions bud through the membranes of the Golgi apparatus.

Bridgen & Elliott (1996) have established a reverse genetics system to generate infectious Bunyamwera virus entirely from cloned cDNAs. They followed a strategy first described by Schnell et al. (1994) for rabies virus: intracellular transcription of a cDNA coding for the positive-sense antigenomic RNA (but not for the negative-sense genomic RNA) in cells expressing the viral polymerase and nucleoprotein. Bridgen & Elliott (1996) infected HeLaT4+ cells with vaccinia virus expressing T7 polymerase and transfected these cells with plasmids expressing proteins encoded by the S, M, and L segments. They then transfected these cells with three plasmids encoding full-length anti-genomic cDNAs flanked by the T7 polymerase promoter and the hepatitis delta virus ribozyme. To increase the number of bunyavirus particles relative to the number of vaccinia virus particles, the authors used mosquito cells in which Bunyamwera but not Vaccinia virus replicates. This protocol can be used not only to genetically engineer Bunyaviridae, but also generate reassortant viruses that cannot easily be obtained by coinfecting cells with different Bunyaviridae strains.

To study bunyavirus promoter elements and the viral proteins that are required for transcription and replication, Dunn et al. (1995) cloned the CAT gene in the negative-sense orientation between the 5′ and 3′ nontranslated regions of the Bunyamwera S RNA segment. Cells were transfected with constructs expressing the proteins encoded by the L and S segment and were then transfected with in vitro transcribed RNA, which resulted in CAT activity. The bunyavirus S segment encodes two proteins, N and NSs, in overlapping reading frames. To determine whether both of these proteins are required for transcription and replication, constructs expressing only N or NSs were tested for CAT activity. N protein expression, together with L protein, resulted in CAT activity, whereas no CAT activity was detected with the NSs expression construct. Thus, the L and N proteins are sufficient for transcription and replication of a bunyavirus-like RNA.

As with influenza virus, the terminal sequences of bunyavirus RNAs are complementary and highly conserved. It has therefore been assumed that these sequence elements define the bunyaviral promoter and are crucial for promoter activity. Deletion of five nucleotides at the 3′ end of the viral RNA drastically reduces CAT expression (Dunn et al., 1995). In contrast, addition of two nucleotides at the 5′ end, or of 11 or 35 nucleotides at the 3′ end does not abolish CAT expression (Dunn et al., 1995). Therefore, like the influenza virus polymerase complex, the bunyavirus polymerase protein can apparently start transcription and/or replication internally.

### Exemplary Virus Vectors

The present invention provides isolated vectors (polynucleotides) which may include tandem transcription units (transcription cassettes) on one or more vectors, e.g., plasmids or other, e.g., linear, nucleic acid delivery vehicles. For example, the compositions of the invention may include one, two, three, four, or more plasmids having virus or reporter genes. In one embodiment, two or more transcription cassettes are combined on one plasmid. This approach allows for efficient and robust expression of multiple genes in cells that generally are not permissive for efficient transfection.

For instance, the invention includes a vector such as a plasmid including tandem transcription cassettes such as one or more of the following cassettes (i) a RNA PolI promoter, a cDNA for a reporter (in negative- or positive-sense orientation), and a RNA Poll terminator; or (ii) a RNA PolII promoter, a DNA for influenza virus PB2, PB1, PA, and/or NP protein, and a polyadenylation signal (RNA PolII transcription termination sequence). In one embodiment, four RNA PolII transcription cassettes for the synthesis of the polymerase and NP proteins are combined on one plasmid. In a further embodiment, three RNA PolII transcription cassettes for the synthesis of the polymerase proteins are combined on one plasmid, and one RNA PolII transcription cassette for NP protein is on another plasmid.

In one embodiment, one or more vectors for vRNA production comprise a promoter including, but not limited to, a RNA PolI promoter, e.g., a human RNA PolI promoter, a RNA PolII promoter, a RNA PolIII promoter, a T7 promoter, or a T3 promoter. For a vector for vRNA which includes a RNA PolII promoter, the vector may optionally include ribozyme sequences (see PCT/US04/016649, the disclosure of which is incorporated by reference herein). Preferred transcription termination sequences for the vRNA vectors include, but are not limited to, a RNA PolI transcription termination sequence, a RNA PolIII transcription termination sequence, or a ribozyme. Each RNA PolII promoter in a plasmid or a combination of plasmids to be employed together, may be the same or different. Each RNA PolI promoter in a plasmid or a combination of plasmids to be employed together, may be the same or different. Likewise, each RNA PolII transcription termination sequence in a plasmid or a combination of plasmids to be employed together, may be the same or different. Further, each RNA PolI transcription termination sequence in a plasmid or a combination of plasmids to be employed together, may be the same or different.

The invention will be further described by the following nonlimiting examples.

### EXAMPLE 1

### Materials and Methods

Cells and viruses. 293T human embryonic kidney cells and Madin-Darby canine kidney cells (MDCK) were maintained in Dulbecco's modified Eagle medium (DMEM) supplemented with 10% fetal calf serum and in modified Eagle's medium (MEM) containing 5% newborn calf serum, respectively. All cells were maintained at 37° C. in 5% CO₂. Influenza viruses A/WSN/33 (H1N1) and A/PR/8/34 (H1N1) were propagated in 10-day-old eggs.

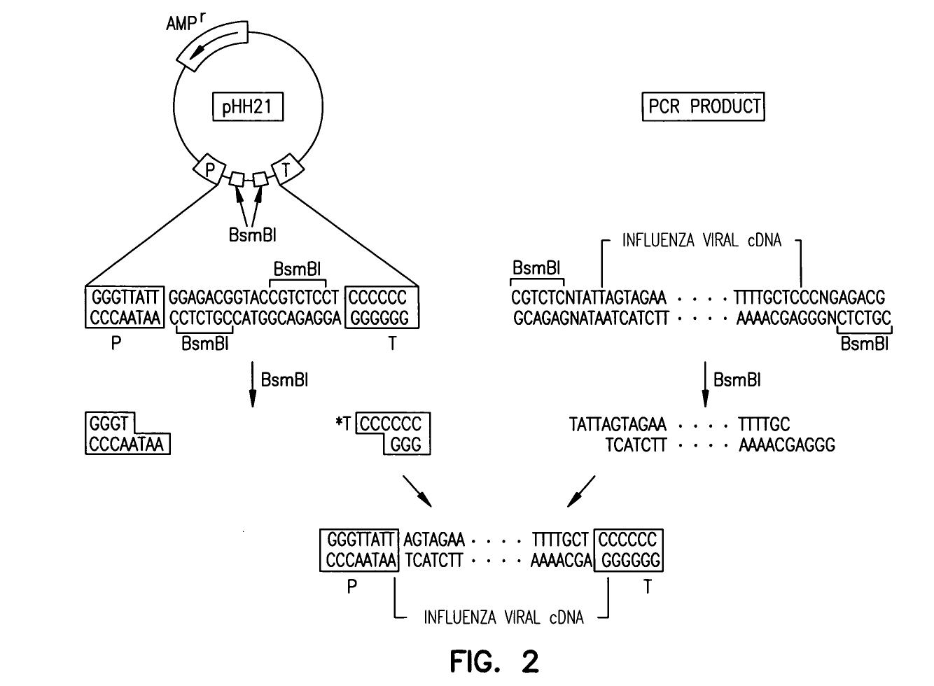

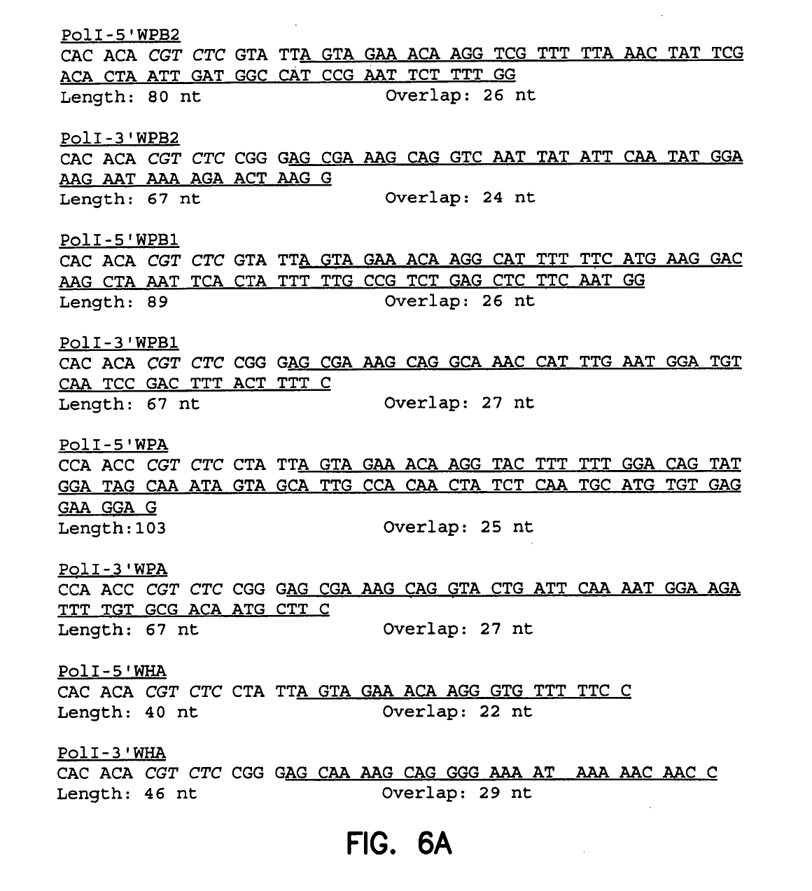

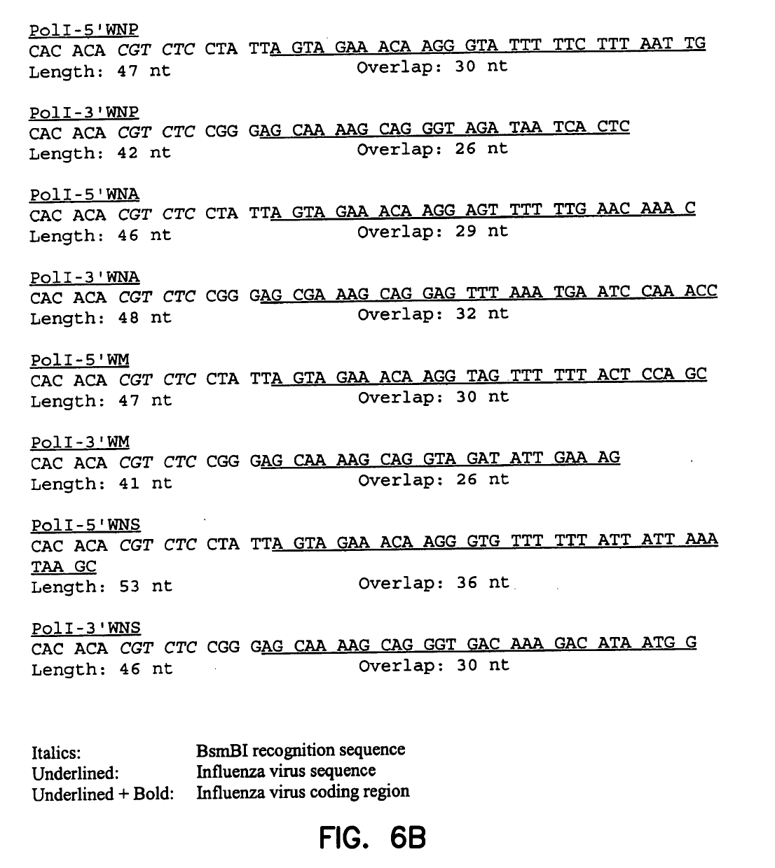

Construction of plasmids. To generate RNA polymerase I constructs, cloned cDNAs derived from A/WSN/33 or A/PR/8/34 viral RNA were introduced between the promoter and terminator sequences of RNA polymerase I. Briefly, the cloned cDNAs were amplified by PCR with primers containing BsmBI sites, digested with BsmBI, and cloned into the BsmBI sites of the pHH21 vector which contains the human RNA polymerase I promoter and the mouse RNA polymerase I terminator, separated by BsmBI sites (FIG. 2). The PB2, PB1, PA, HA, NP, NA, M, and NS genes of the A/WSN/33 strain were PCR-amplified by use of the following plasmids: pSCWPB2, pGW-PB1, and pSCWPA (all obtained from Dr. Debi Nayak at the University of California Los Angeles), and pWH17, pWNP152, pT3WNA15 (Castrucci et al., 1992), pGT3WM, and pWNS1, respectively. The PB1 gene of influenza A/PR/8/34 virus was amplified by using pcDNA774 (PB1) (Perez et al., 1998) as a template. See FIG. 6 for the sequences of the primers. To ensure that the genes were free of unwanted mutations, PCR-derived fragments were sequences with an autosequencer (Applied Biosystem Inc., CA, USA) according to the protocol recommended by the manufacturer. The cDNAs encoding the HA, NP, NA, and M1 genes of A/WSN/33 virus were cloned as described (Huddleston et al., 1982) and subcloned into the eukaryotic expression vector pCAGGS/MCS (controlled by the chicken β-actin promoter) (Niwa et al., 1991), resulting in pEWSN-HA, pCAGGS-WSN-NP0-14, pCAGGS-WNA15, and pCAGGS-WSN-M1-2/1, respectively. The M2 and NS2 genes from the A/PR/8/34 virus were amplified by PCR and cloned into pCAGGS/MCS, yielding pEP24c and pCA-NS2. Finally, pcDNA774(PB1), pcDNA762(PB2), and pcDNA787(PA) were used to express the PB2, PB1, and PA proteins under control of the cytomegalovirus promoter (Perez et al., 1998).

Generation of infectious influenza particles. 293T cells (1×10⁶) were transfected with a maximum of 17 plasmids in different amounts with use of Trans IT LT-1 (Panvera, Madison, Wis.) according to the manufacturer's instructions. Briefly, DNA and transfection reagent were mixed (2 μl Trans IT-LT-1 per μg of DNA), incubated at room temperature for 45 minutes and added to the cells. Six hours later, the DNA-transfection reagent mixture was replaced by Opti-MEM (Gibco/BRL, Gaithersburg, Md.) containing 0.3% bovine serum albumin and 0.01% fetal calf serum. At different times after transfection, viruses were harvested from the supernatant and titrated on MDCK cells. Since helper virus was not required by this procedure, the recovered transfectant viruses were analyzed without plaque purification.

Determination of the percentage of plasmid-transfected cells producing viruses. Twenty-four hours after transfection, 293T cells were dispersed with 0.02% EDTA into single cells. The cell suspension was then diluted 10-fold and transferred to confluent monolayers of MDCK cells in 24-well plates. Viruses were detected by the hemagglutination assay.

Immunostaining assay. Nine hours after infection with influenza virus, cells were washed twice with phosphate-buffered saline (PBS) and fixed with 3.7% paraformaldehyde (in PBS) for 20 minutes at room temperature. Next, they were treated with 0.1% Triton X-100 and processed as described by Neumann et al. (1997).

### Results

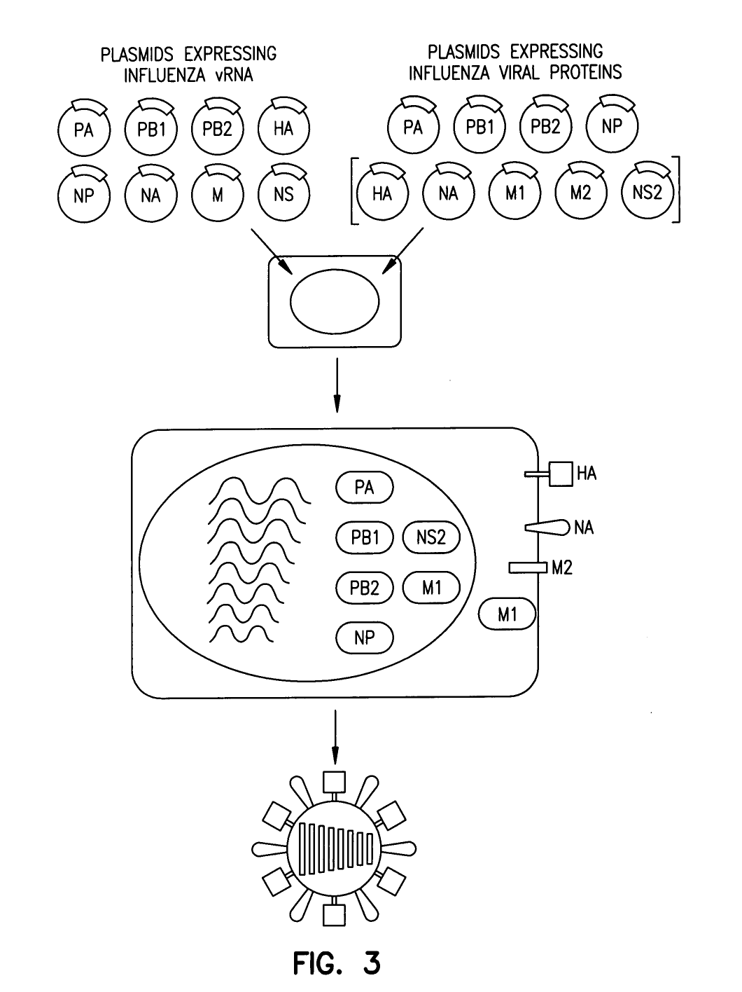

Generation of infectious virus by plasmid-driven expression of viral RNA segments, three polymerase subunits and NP protein. Although transfection of cells with a mixture of RNPs extracted from purified virions results in infectious influenza particles, this strategy is not likely to be efficient when used with eight different in vitro generated RNPs. To produce infectious influenza viruses entirely from cDNAs, eight viral RNPs were generated in vivo. Thus, plasmids were prepared that contain cDNAs for the full-length viral RNAs of the A/WSN/33 virus, flanked by the human RNA polymerase I promoter and the mouse RNA polymerase I terminator. In principle, transfection of these eight plasmids into eukaryotic cells should result in the synthesis of all eight influenza vRNAs. The PB2, PB1, PA and NP proteins, generated by cotransfection of protein expression plasmids, should then assemble the vRNAs into functional vRNPs that are replicated and transcribed, ultimately forming infectious influenza viruses (FIG. 3). 1×10⁶ 293T cells were transfected with protein expression plasmids (1 μg of pcDNA762(PB2), 1 μg of pcDNA774(PB1), 0.1 μg of pcDNA787(PA), and 1 μg of pCAGGS-WSN-NP0/14) and 1 μg of each of the following RNA polymerase I plasmids (pPo1I-WSN-PB2, pPo1I-WSN-PB1, pPo1I-WSN-PA, pPo1I-WSN-HA, pPo1I-WSN-NP, pPo1I-WSN-NA, pPo1I-WSN-M, and pPo1I-WSN-NS). The decision to use a reduced amount of pcDNA787(PA) was based on previous observations (Mena et al., 1996), and data on the optimal conditions for generation of virus-like particles (VLPs) (data not shown). Twenty-four hours after transfection of 293T cells, 7×10³ pfu of virus per ml was found in the supernatant (Experiment 1, Table 1), demonstrating for the first time the capacity of reverse genetics to produce influenza A virus entirely from plasmids.

TABLE 1
Plasmid sets used to produce influenza virus from cloned cDNA*

| | Experiment | | | | | | | |
|---|---|---|---|---|---|---|---|---|
| | 1 | 2 | 3 | 4 | 5 | 6 | 7 | 8 |
| RNA polymerase I | | | | | | | | |
| plasmids for:^{†} | | | | | | | | |
| PB1 | + | + | − | − | − | − | − | − |
| PR8-PB1 | − | − | + | + | + | + | + | + |
| PB2 | + | + | + | + | + | + | + | + |
| PA | + | + | + | + | + | + | + | + |
| HA | + | + | + | + | + | + | + | + |
| NP | + | + | + | + | + | + | + | + |
| NA | + | + | + | + | + | + | + | + |
| M | + | + | + | + | + | + | + | + |
| NS | + | + | + | + | + | + | + | + |
| Protein expression | | | | | | | | |
| plasmids for: | | | | | | | | |
| PB1 | + | + | + | + | − | + | + | + |
| PB2 | + | + | + | + | + | − | + | + |
| PA | + | + | + | + | + | + | − | + |
| NP | + | + | + | + | + | + | + | − |
| HA | − | + | − | + | + | + | + | + |
| NA | − | + | − | + | + | + | + | + |
| M1 | − | + | − | + | + | + | + | + |
| M2 | − | + | − | + | + | + | + | + |
| NS2 | − | + | − | + | + | + | + | + |
| Virus titer (pfu/ml) | 7 × 10³ | 7 × 10³ | 1 ×10³ | 3 × 10⁴ | 0 | 0 | 0 | 0 |

*293T cells were transfected with the indicated plasmids. Twenty-four (Experiments 1 and 2) or forty-eight hours (Experiments 3-8) later, the virus titer in the supernatant was determined in MDCK cells.
^{†}Unless otherwise indicated, plasmids were constructed with cDNAs representing the RNAs of A/WSN/33 virus.

Efficiency of influenza virus production with coexpression of all viral structural proteins. Although expression of the viral NP and polymerase proteins is sufficient for the plasmid-driven generation of influenza viruses, it was possible that the efficiency could be improved. In previous studies, the expression of all influenza virus structural proteins (PB2, PB1, PA, HA, NP, NA, M1, M2, and NS2) resulted in VLPs that contained an artificial vRNA encoding a reporter chloramphenicol-acetyltransferase gene (Mena et al., 1996). Thus, the availability of the entire complement of structural proteins, instead of only those required for viral RNA replication and transcription, might improve the efficiency of virus production. To this end, 293T cells were transfected with optimal amounts of viral protein expression plasmids (as judged by VLP production; unpublished data): 1 μg of pcDNA762(PB2) and pcDNA774(PB1); 0.1 μg of pcDNA787(PA); 1 μg of pEWSN-HA, pCAGGS-WSN-NP0/14, and pCAGGS-WNA15; 2 μg of pCAGGS-WSN-M1-2/1; 0.3 μg of pCA-NS2; and 0.03 μg of pEP24c (for M2), together with 1 μg of each RNA polymerase I plasmid (Experiment 2, Table 1). A second set of cells was transfected with the same set of RNA polymerase I plasmids, with the exception of the PB1 gene, for which pPo1I-PR/8/34-PB1 was substituted in an effort to generate a reassortant virus, together with plasmids expressing only PA, PB1, PB2, and NP (Experiment 3, Table 1) or those expressing all the influenza structural proteins (Experiment 4, Table 1). Yields of WSN virus did not appreciably differ at 24 hours (Experiments 1 and 2, Table 1) or at 36 hours (data not shown) post-transfection. However, more than a 10-fold increase in yields of the virus with PR/8/34-PB1 was found when all the influenza viral structural proteins were provided (Experiments 3 and 4, Table 1). Negative controls, which lacked one of the plasmids for the expression of PA, PB1, PB2, of NP proteins, did not yield any virus (Experiments 5-8, Table 1). Thus, depending on the virus generated, expression of all influenza A virus structural proteins appreciably improved the efficiency of the reverse genetics method.

Next, the kinetics of virus production after transfection of cells was determined using the set of plasmids used to generate a virus with the A/PR/8/34-PB1 gene. In two of three experiments, virus was first detected at 24 hours after transfection. The titer measured at that time, >10³ pfu/ml, had increased to >10⁶ pfu/ml by 48 hours after transfection (Table 2). To estimate the percentage of plasmid-transfected cells that were producing viruses, 293T cells were treated with EDTA (0.02%) at 24 hours after transfection to disperse the cells, and then performed limiting dilution studies. In this experiment, no free virus was found in the culture supernatant at this time point. The results indicated that 1 in 10^{3.3} cells was generating infectious virus particles.

TABLE 2
Kinetics of virus production after plasmid
transfection into 293T cells*

| Hours after | Virus titers in culture supernatant (pfu/ml) | | |
|---|---|---|---|
| plasmid | Experiment | | |
| transfection | 1 | 2 | 3 |
| 6 | 0 | ND | ND |
| 12 | 0 | ND | 0 |
| 18 | 0 | ND | 0 |
| 24 | 0 | 2 × 10³ | 6 × 10³ |
| 30 | ND | 5 × 10⁴ | 9 × 10⁴ |
| 36 | 6 × 10² | >1 × 10⁵ | 7 × 10⁵ |
| 42 | ND | >1 × 10⁶ | 5 × 10⁶ |
| 48 | 8 × 10⁴ | >1 × 10⁶ | 1 × 10⁷ |

*293T cells were transfected with eight RNA polymerase I plasmids encoding A/WSN/33 virus genes with the exception of PB1 gene, which is derived from A/PR/8/34 virus, and nine protein expression plasmids as described in the text. At different time points, we titrated virus in the culture supernatant in MDCK cells.
ND = not done.

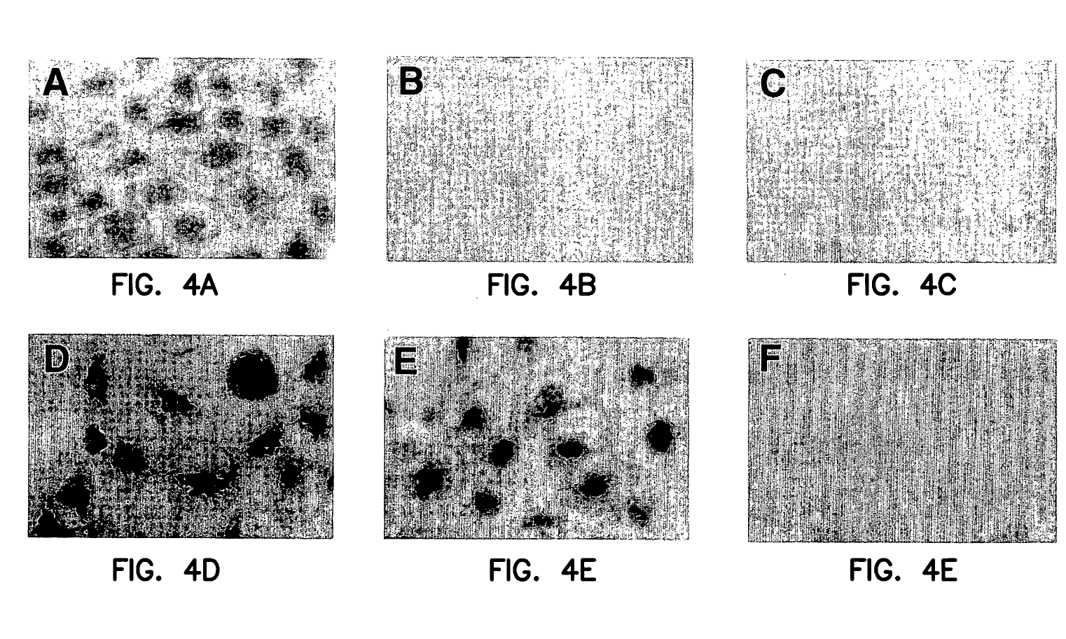

Recovery of influenza virus containing the FLAG epitope in the NA protein. To verify that the new reverse genetics system allowed the introduction of mutations into the genome of influenza A viruses, a virus containing a FLAG epitope (Castrucci et al., 1992) in the NA protein was generated. 293T cells were transfected with an RNA polymerase I plasmid (pPo1I-WSN-NA/FL79) that contained a cDNA encoding both the NA protein and a FLAG epitope at the bottom of the protein's head, together with the required RNA polymerase I and protein expression plasmids. To confirm that the recovered virus (PR8-WSN-FL79) did in fact express the NA-FLAG protein, immunostaining assays of cells infected with PR8-WSN-FL79 or A/WSN/33 wild-type virus was performed. A monoclonal antibody to the FLAG epitope detected cells infected with PR8-WSN-FL79, but not those infected with wild-type virus (FIG. 4). Recovery of the PR8-WSN-FL79 virus was as efficient as that for the untagged wild-type virus (data not shown). These results indicate that the new reverse genetics system allows one to introduce mutations into the influenza A virus genome.

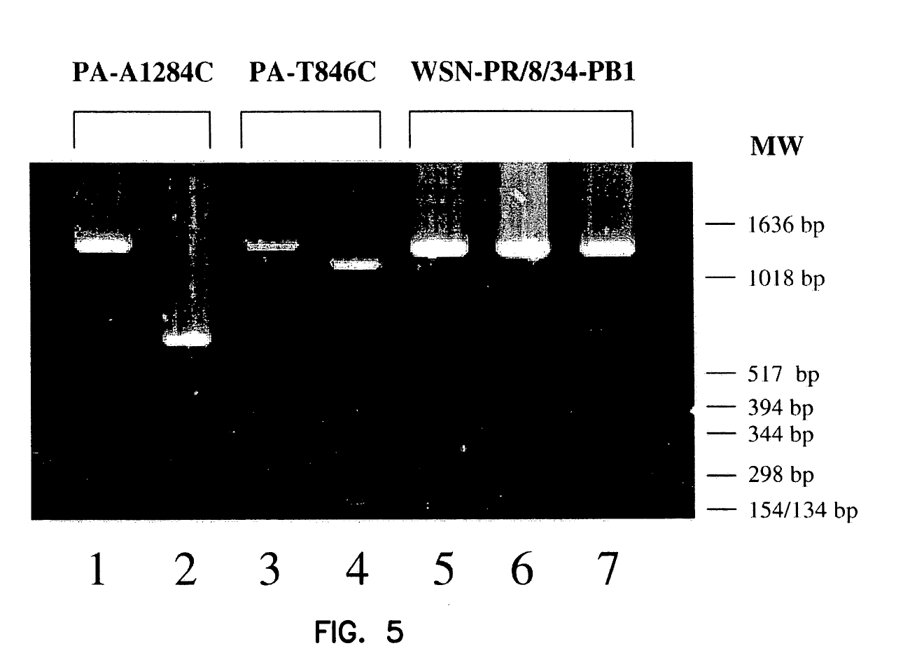

Generation of infectious influenza virus containing mutations in the PA gene. To produce viruses possessing mutations in the PA gene, two silent mutations were introduced creating new recognition sequences for restriction endonucleases (Bsp120I at position 846 and PvuII at position 1284 of the mRNA). Previously, it was not possible to modify this gene by reverse genetics, because of the lack of a reliable selection system. Transfectant viruses, PA-T846C and PA-A1284 were recovered. The recovered transfectant viruses were biologically cloned by two consecutive limiting dilutions. To verify that the recovered viruses were indeed transfectants with mutations in the PA gene, cDNA for the PA gene was obtained by reverse transcriptase-PCR. As shown in FIG. 5, PA-T846C and PA-A1284C viruses had the expected mutations within the PA gene, as demonstrated by the presence of the newly introduced restriction sites. PCR of the same viral samples and primers without the reverse transcription step failed to produce any products (data not shown), indicating that the PA cDNA was indeed originated from vRNA instead of the plasmid used to generate the viruses. These results illustrate how viruses with mutated genes can be produced and recovered without the use of helper viruses.

### Discussion

The reverse genetics systems described herein allows one to efficiently produce influenza A viruses entirely from cloned cDNAs. Bridgen and Elliott (1996) also used reverse genetics to generate a Bunyamwera virus (Bunyaviridae family), but it contains only three segments of negative-sense RNA, and the efficiency of its production was low, 10² pfu/10⁷ cells. Although the virus yields differed among the experiments, consistently >10³ pfu/10⁶ cells was observed for influenza virus, which contains eight segments. There are several explanations for the high efficiency of the reverse genetics system described hereinabove. Instead of producing RNPs in vitro (Luytjes et al., 1989), RNPs were generated in vivo through intracellular synthesis of vRNAs using RNA polymerase I and through plasmid-driven expression of the viral polymerase proteins and NP. Also, the use of 293T cells, which are readily transfected with plasmids (Goto et al., 1997), ensured that a large population of cells received all of the plasmids needed for virus production. In addition, the large number of transcripts produced by RNA polymerase I, which is among the most abundantly expressed enzymes in growing cells, likely contributed to the overall efficiency of the system. These features led to a correspondingly abundant number of vRNA transcripts and adequate amounts of viral protein for encapsidation of vRNA, formation of RNPs in the nucleus, and export of these complexes to the cell membrane, where new viruses are assembled and released.

Previously established reverse genetics systems (Enami et al., 1990; Neumann et al., 1994; Luytjes et al., 1989; Pleschka et al., 1996) require helper-virus infection and therefore selection methods that permit a small number of transfectants to be retrieved from a vast number of helper viruses. Such strategies have been employed to generate influenza viruses that possess one of the following cDNA-derived genes: PB2 (Subbarao et al., 1993), HA (Enami et al., 1991: Horimoto et al., 1994), NP (Li et al., 1995), NA (Enami et al., 1990), M (Castrucci et al., 1995; Yasuda et al., 1994), and NS (Enami et al., 1991). Most of the selection methods, except for those applicable to the HA and NA genes, rely on growth temperature, host range restriction, or drug sensitivity, thus limiting the utility of reverse genetics for functional analysis of the gene products. Even with the HA and NA genes, for which reliable antibody-driven selection systems are available, it is difficult to produce viruses with prominent growth defects. In contrast, the reverse genetics system described herein does not require helper virus and permits one to generate transfectants with mutations in any gene segment or with severe growth defects. This advantage is demonstrated in FIG. 5, which the recovery of transfectant viruses with a mutated PA gene. Having the technology to introduce any viable mutation into the influenza A virus genome will enable investigators to address a number of long-standing issues, such as the nature of regulatory sequences in nontranslated regions of the viral genome, structure-function relationships of viral proteins, and the molecular basis of host-range restriction and viral pathogenicity.

Although inactivated influenza vaccines are available, their efficacy is suboptimal due partly to their limited ability to elicit local IgA and cytotoxic T cell responses. Clinical trials of cold-adapted live influenza vaccines now underway suggest that such vaccines are optimally attenuated, so that they will not cause influenza symptoms, but will still induce protective immunity (reviewed in Keitel & Piedra, 1998). However, preliminary results indicate that these live virus vaccines will not be significantly more effective than the best inactivated vaccine (reviewed in Keitel. & Piedra, 1998), leaving room for further improvement. One possibility would be to modify a cold-adapted vaccine with the reverse genetics system described above. Alternatively, one could start from scratch by using reverse genetics to produce a “master” influenza A strain with multiple attenuating mutations in the genes that encode internal proteins. The most intriguing application of the reverse genetics system described herein may lie in the rapid production of attenuated live-virus vaccines in cases of suspected pandemics involving new HA or NA subtypes of influenza virus.

This new reverse genetics system will likely enhance the use of influenza viruses as vaccine vectors. The viruses can be engineered to express foreign proteins or immunogenic epitopes in addition to the influenza viral proteins. One could, for example, generate viruses with foreign proteins as a ninth segment (Enami et al., 1991) and use them as live vaccines. Not only do influenza viruses stimulate strong cell-mediated and humoral immune responses, but they also afford a wide array of virion surface HA and NA proteins (e.g., 15 HA and 9 NA subtypes and their epidemic variants), allowing repeated immunization of the same target population.

Influenza VLPs possessing an artificial vRNA encoding a reporter gene have been produced by expressing viral structural proteins and vRNA with the vaccinia-T7 polymerase system (Mena et al., 1996). Using reverse genetics, one can now generate VLPs containing vRNAs that encode proteins required for vRNA transcription and replication (i.e., PA, PB1, PB2, and NP), as well as vRNAs encoding proteins of interest. Such VLPs could be useful gene delivery vehicles. Importantly, their lack of genes encoding viral structural proteins would ensure that infectious viruses will not be produced after VLP-gene therapy. Since the influenza virus genome is not integrated into host chromosome, the VLP system would be suitable for gene therapy in situations requiring only short-term transduction of cells (e.g., for cancer treatment). In contrast to adenovirus vectors (Kovesdi et al., 1997), influenza VLPs could contain both HA and NA variants, allowing repeated treatment of target populations.

The family Orthomyxoviridae comprises influenza A, B, and C viruses, as well as the recently classified Thogotovirus. The strategy for generating infectious influenza A viruses entirely from cloned cDNAs described herein would apply to any orthomyxovirus, and perhaps to other segmented negative-sense RNA viruses as well (e.g., Bunyaviridae, Arenaviridae). The ability to manipulate the viral genome without technical limitations has profound implications for the study of viral life cycles and their regulation, the function of viral proteins and the molecular mechanisms of viral pathogenicity.

### EXAMPLE 2

### Expression of the Influenza Virus Proteins PB2, PB1, PA, and NP Leads to Replication and Transcription of an Artificial Viral RNA

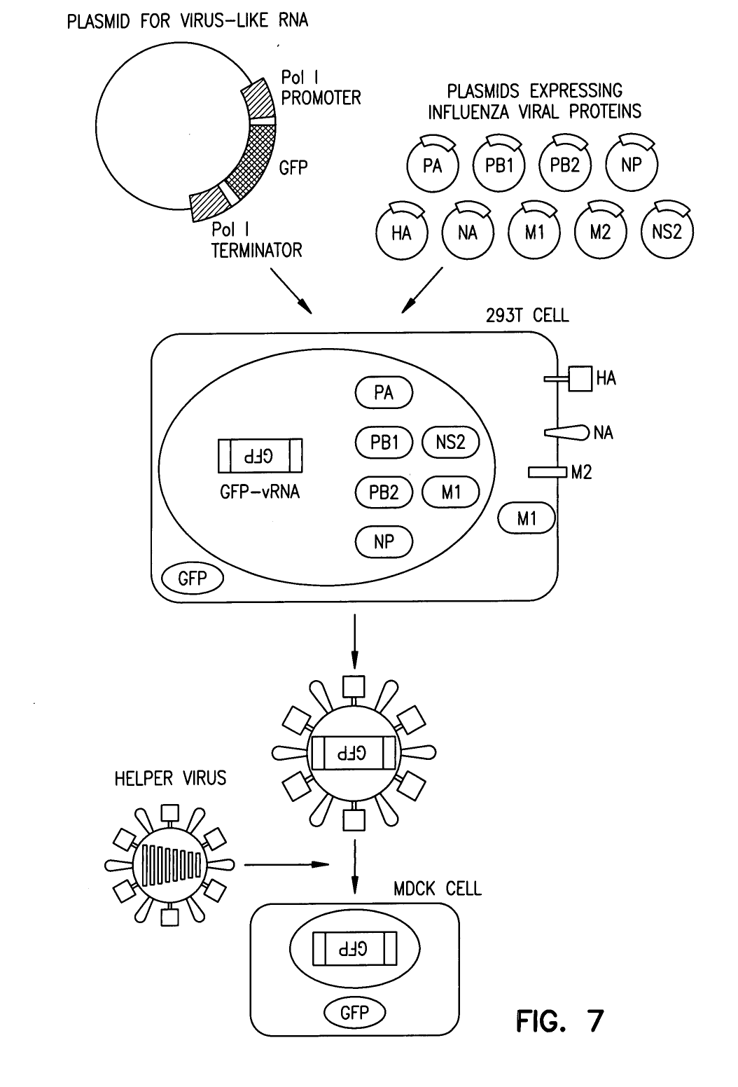

To generate influenza VLPs, the RNA polymerase I system for the intracellular synthesis of influenza viral RNAs in vivo was employed (FIG. 7). In this system, a cDNA encoding a reporter gene in antisense orientation is flanked by the 5′ and 3′ noncoding regions of an influenza viral RNA. This cassette is inserted between an RNA polymerase I promoter and terminator. Transfection of such constructs into eukaryotic cells leads to transcription of the reporter gene by cellular RNA polymerase I, thereby generating influenza virus-like RNAs (Neumann et al., 1994). Upon influenza virus infection, the artificial vRNAs are replicated and transcribed by the viral polymerase complex, resulting in the expression of the reporter gene.

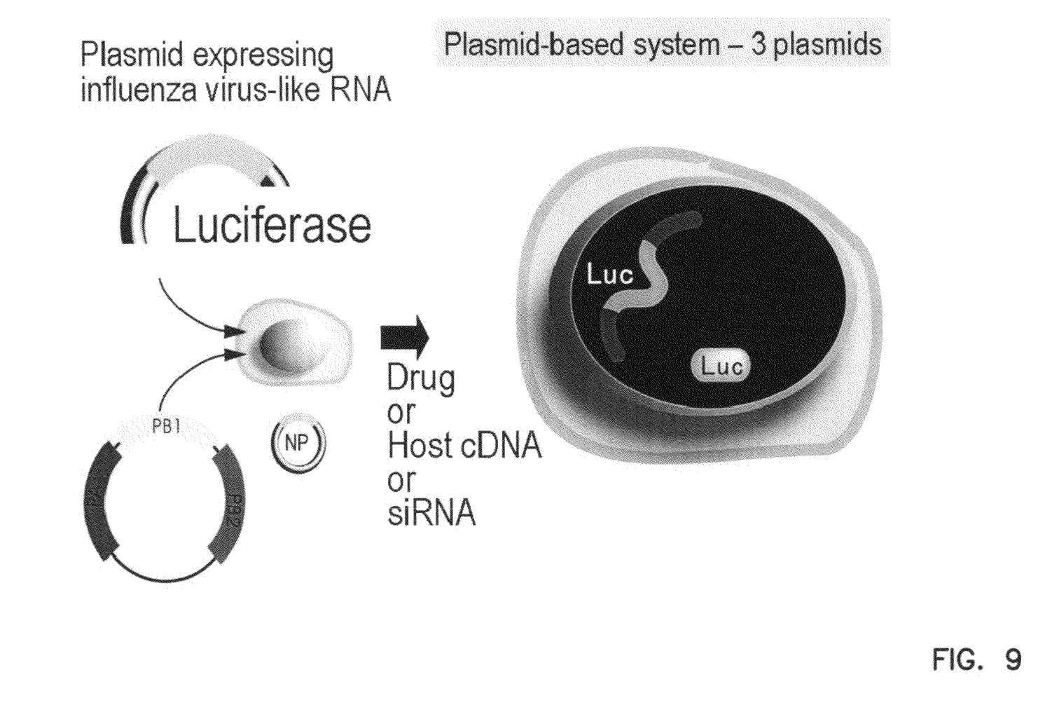

To determine whether expression of the PB2, PB1, PA, and NP proteins leads to expression of the reporter gene encoded by the RNA polymerase I-derived transcript, plasmids (1 μg each) expressing the NP protein of A/WSN/33 (H1N1) virus under control of the chicken β-actin promoter (pCAGGS-WSN-NP0/14), the polymerase proteins of A/PR/8/34 virus under control of the cytomegalovirus promoter [pcDNA762(PB2), pcDNA774(PB1), and pcDNA787(PA)], and an RNA polymerase I reporter gene construct (pPolI-GFP) were transfected into human embryonic kidney (293T) cells. Forty eight hours later, 30%-40% of the cells were expressing GFP (FIG. 9). In contrast, GFP expression could not be detected in transfected cells lacking the polymerase or NP proteins. These results indicated that NP and the three influenza viral polymerase proteins had formed a functional complex that replicated and transcribed the RNA polymerase I-derived GFP vRNA.

Optimal vRNA transcription and replication. To determine the amounts of plasmid DNA required for optimal reporter GFP expression, the expression of the polymerase proteins and NP was modulated. Previous studies had indicated that large amounts of PA reduce the extent of reporter gene expression in transcription/replication systems (Mena et al., 1996). Therefore, in a stepwise manner, the expression of PA from the plasmid was reduced, identifying 0.1 μg of pcDNA787(PA) as the template amount yielding the strongest expression of GFP. With NP, the major structural component of RNP complexes, high amounts of protein expression plasmid may be required. However, higher amounts of the plasmid did not appreciably affect the number of GFP-positive 293T cells. In addition, various amounts of the PB2 and PB1 protein expression plasmids (ranging from 1.0 to 0.03 μg) did not affect the GFP expression in 293T cells. Hence, in all subsequent experiments, 0.1 μg of pcDNA787(PA), and 1.0 μg of pcDNA774(PB1), pcDNA762(PB2), and pCAGGS-WSN-NP0/14, was used.

Formation of influenza VLPs from cloned cDNAs. Previous studies with the vaccinia virus T7 RNA polymerase system showed that the formation of influenza VLPs requires nine influenza virus proteins: PB2, PB1, PA, HA, NA, NP, M1, M2, and NS2 (Mena et al., 1996). The NS1 protein, by contrast, is dispensable for particle formation (Mena et al., 1996). To establish an efficient plasmid-driven system for VLP generation, cDNAs were generated that encoded the HA, NA, M1, M2, and NS2 genes. The cDNAs were cloned into the eukaryotic expression vector pCAGGS/MCS (controlled by the chicken β-actin promoter), resulting in pEWSN-HA, pCAGGS-WNA15, pCAGGS-WSN-M1-2/1, pEP24c, and pCA-NS2, respectively. Expression of each protein was confirmed by Western blot analysis.

To generate VLPs, 10⁶ 293T cells were transfected with 1.0 μg of each protein expression plasmid (with the exception of pcDNA787(PA), for which 0.1 μg was employed), and with 1 μg of the reporter gene construct pPolI-GFP. Culture supernatants were harvested 48 hours after transfection and mixed with A/WSN/33 virus to provide the influenza virus proteins required for replication and transcription of GFP vRNA. The mixture was then inoculated into MDCK cells. Ten hours after incubation, GFP-positive MDCK cells were detected, corresponding to 450 particles/ml of supernatant (Table 3). Thus, plasmid-driven expression of all influenza viral structural proteins resulted in the formation of infectious influenza VLPs containing GFP vRNA. Moreover, GFP vRNA was delivered to MDCK cells.

Optimal assembly of influenza virus. VLP formation was also studied in cells expressing different amounts of the RNA polymerase I reporter gene construct, as well as HA, NA, M1, M2, and NS2 plasmid DNAs. In experiments with pPolI-GFP, 1.0 μg of the plasmid DNA was highly efficient in generating VLPs, whereas the efficiency was significantly reduced for 2.0 μg or 3.0 μg. Because the NS2 and M2 proteins are expressed in low amounts late in infection, it was likely that relatively small amounts of the expression plasmids would be needed for optimal VLP formation. Reduction of the M2 expression construct from 1.0 μg to 0.3 μg resulted in a more than tenfold increase in the number of GFP-positive MDCK cells (Table 3). Further reduction of plasmid to 0.03 μg did not increase the number of VLPs. For NS2, lower amounts of plasmid tested (0.1 μg) were associated with less efficient formation of VLPs (Table 3).

The M1 protein is the major structural component of the virion. Thus, high levels of M1 expression are likely required for efficient formation of VLPs. This prediction was tested in experiments comparing VLP formation in cells transfected with 1.0 μg or 2.0 μg of M1 plasmid DNA. As shown in Table 3, higher amounts of plasmid resulted in a more than tenfold increase in the number of GFP-positive MDCK cells. Comparison of two different amounts (1 μg vs. 2 μg) of plasmids expressing the HA and NA proteins did not reveal any appreciable differences in VLP formation, leading to selection of 1 μg of each plasmid (pEWSN-HA, pCAGGS-WNA15) for use in subsequent experiments. Overall, these studies resulted in a >100-fold increase in the efficiency of VLP formation, ultimately leading to the production of more than 104 infectious influenza virus particles per ml of supernatant.

TABLE 3
Optimal amounts of plasmid DNA for the formation of infectious VLPs*

| Amount (μg) of plasmid DNA expressing: | | | | | | | | | | |
|---|---|---|---|---|---|---|---|---|---|---|
| | | | | | | | | | GFP | Relative efficiency of |
| PB2 | PB1 | PA | HA | NP | NA | M1 | M2 | NS2 | vRNA | VLP formation⁺ |
| 1.0 | 1.0 | 0.1 | 1.0 | 1.0 | 1.0 | 1.0 | 1.0 | 1.0 | 1.0 | 1 |
| 1.0 | 1.0 | 0.1 | 1.0 | 1.0 | 1.0 | 1.0 | 0.1 | 1.0 | 1.0 | 28 |
| 1.0 | 1.0 | 0.1 | 1.0 | 1.0 | 1.0 | 1.0 | 0.03 | 1.0 | 1.0 | 17 |
| 1.0 | 1.0 | 0.1 | 1.0 | 1.0 | 1.0 | 1.0 | 0.1 | 1.0 | 1.0 | 28 |
| 1.0 | 1.0 | 0.1 | 1.0 | 1.0 | 1.0 | 1.0 | 0.1 | 0.3 | 1.0 | 24 |
| 1.0 | 1.0 | 0.1 | 1.0 | 1.0 | 1.0 | 1.0 | 0.1 | 0.1 | 1.0 | 11 |
| 1.0 | 1.0 | 0.1 | 1.0 | 1.0 | 1.0 | 1.0 | 0.1 | 1.0 | 1.0 | 28 |
| 1.0 | 1.0 | 0.1 | 1.0 | 1.0 | 1.0 | 2.0 | 0.1 | 1.0 | 1.0 | 220 |

*293T cells were transfected with expression plasmids for all nine influenza virus structural proteins and with the RNA polymerase I-GFP gene plasmid. Forty-eight hours after transfection, VLP-containing supernatants were collected, mixed with A/WSN/33 helper virus, and inoculated into MDCK cells. The cells were fixed 10 hours after infection and GFP expression was determined with a fluorescence microscope. Only the amounts of the M1, M2, and NS2 plasmids were varied (bold letter) to determine their optimal amounts for GFP expression in MDCK cells.
⁺The relative efficiency of VLP formation was determined by counting the number of GFP-positive cells in five microscopic fields. The sample containing 1 μg of each plasmid (which yielded 450 infectious VLP/ml of supernatant) was chosen as the reference (value of 1).

Authenticity of VLPs produced entirely from plasmids. To verify that VLPs initiate infection in the same manner as authentic influenza viruses, VLPs were neutralized with antibody to the WSN HA. VPL-containing supernatants derived from plasmid-transfected 293T cells were incubated with a pool of anti-WSN HA monoclonal antibodies or with a monoclonal antibody to the G protein of vesicular stomatitis virus (VSV) (negative control) for 1 hour at room temperature. A/PR/8/34 helper virus, which is not neutralized by the pool of anti-WSN HA monoclonal antibodies, was added to the mixture and inoculated into MDCK cells. Only the anti-WSN-HA-specific monoclonal antibody neutralized the VLPs, indicating that the HA medicates the attachment and entry of VLPs into cells.

Next, the minimal set of proteins required for the formation of VLPs was identified. Other have established that the three influenza virus polymerases and the NP are essential for the replication and transcription of vRNA (Honda et al., 1988). Therefore, each of these four proteins was included, but HA, NA, M1, M2, or NS2 was consecutively omitted. Exclusion of any of these plasmids did not affect the replication/transcription of GFP vRNA in transfected 293T cells. Supernatants derived from transfected 293T cells that lacked the HA, NA, M1, or NS2 protein did not promote GFP expression in infected MDCK cells, indicating the absence of infectious VLPs. Infectious VLPs were detected with omission of M2 but the number was low (>500 fold reduction compared to the full set of structural proteins). Thus, all influenza virus structural proteins are required for the efficient formation of infectious VLPs, in accord with data from studies of the vaccinia-virus system (Mena et al., 1996).

VSV glycoprotein can replace the HA and NA proteins in the production of VLPs. The influenza virus HA and NA proteins were replaced with the VSV G protein, which functions in receptor binding and fusion. In 293T cells transfected with pPolI-GFP; optimal amounts of the PB2, PB1, PA, NP, M1, M2, and NS2 expression constructs; and 1 μg of the VSV-G construct (pCAGGS-VSV-G), substitution of the VSV-G protein for influenza virus glycoproteins did not adversely affect VLP formation. To the contrary, higher numbers of GFP-positive cells were reproducibly found when VSV-G, rather than the HA and NA, served as the viral glycoprotein. Thus, the VSV G protein can be efficiently incorporated into influenza virions and can function as well as the HA and NA in virus release and entry.

An efficient system for generating infectious influenza virus particles would be an asset in research with this virus and potentially in the production of vaccines and vectors for gene therapy. In contrast to the extant vaccinia virus system, the VLP production strategy described here is highly efficient, both in the initial transfection of cells and in the yield of VLPs (>10⁴ infectious particles/ml of supernatant). Moreover, it is driven entirely by plasmids expressing influenza virus proteins (i.e., in the absence of any other viral proteins), which greatly simplifies the interpretation of results. Another major advantage is the capability to study the effects of lethal mutations in virion formation, packaging of RNP complexes, budding of virus replication, and binding and fusion processes. In addition, it is likely that the system described hereinabove would operate equally well with other viruses, e.g., paramyxoviruses and rhabdoviruses.

Influenza virus HA and NA proteins can be functionally replaced by the VSV glycoprotein G. Previously, it had been reported that influenza viruses failed to incorporate VSV G protein when provided by recombinant SV40 virus (Naim et al., 1993). The results described herein suggest that neither the HA nor the NA is essential for the formation of VLPs, although it cannot be ruled out that these glycoproteins play a role in interactions with other viral proteins, thus affecting the structure of virions, as suggested by the elongated shapes of viruses expressing tail-less HAs, NAs, or both (Garcia-Sastre et al., 1995; Jin et al., 1994; Jin et al., 1997).

The plasmid-based system described hereinabove may be particularly useful for therapeutic gene delivery. VLPs can be prepared that contain the vRNA encoding the proteins required for transcription and replication (i.e., the NP and the polymerases), as well as a vRNA encoding the protein of interest. These particles are infectious and can deliver a designated gene into target cells, where it would replicate and be transcribed. Because these particles do not contain a complete complement of viral genes, they can not produce infectious progeny viruses. This feature, together with the lack of integration of the viral genome into host chromosomes, would ensure the biological safety of gene delivery in human and nonhuman subjects. Finally, the availability of 15 HA and 9 NA subtypes and their variants would allow the repeated administration of VLPs, thereby overcoming immunoresistance to vector-generated proteins, one of the major obstacles faced with repeated use of other viral vectors, such as adenoviruses. A further benefit of the plasmid-driven system would be realized in situations requiring only short-term expression of foreign proteins, as in cancer treatment.

### EXAMPLE 3

### A Method to Screen for Modulators of Viral Transcription or Replication

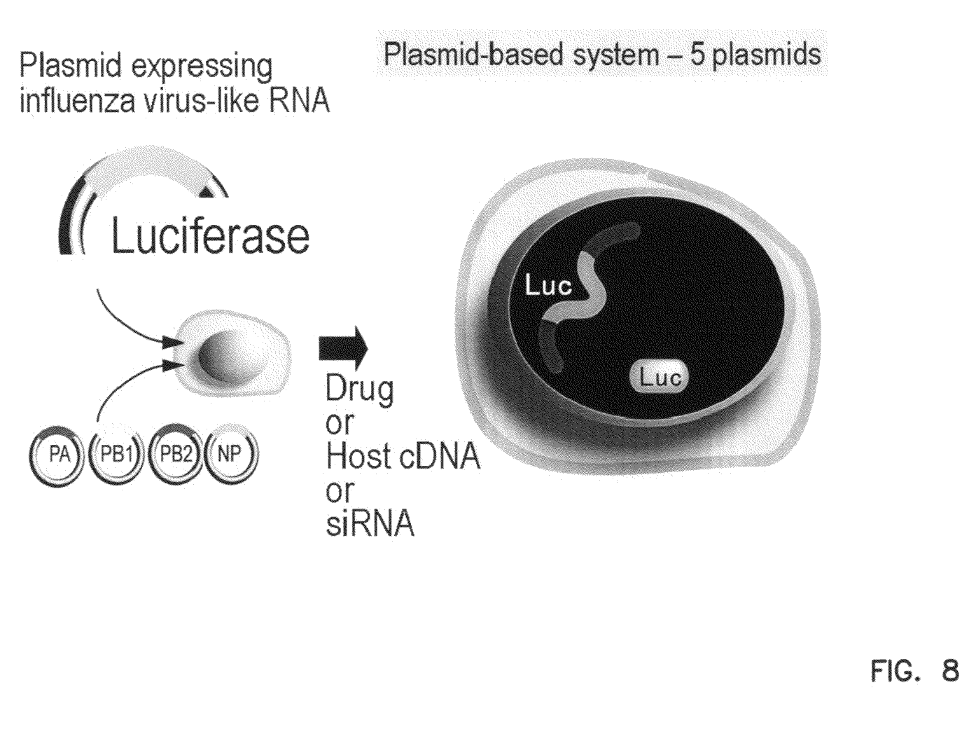

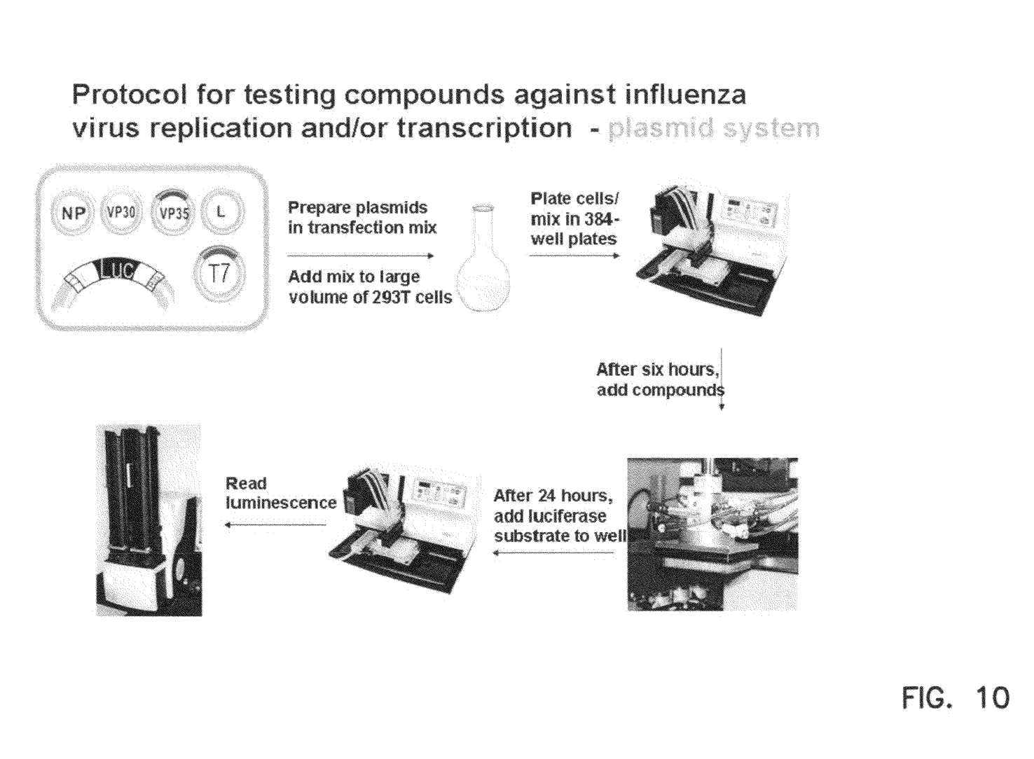

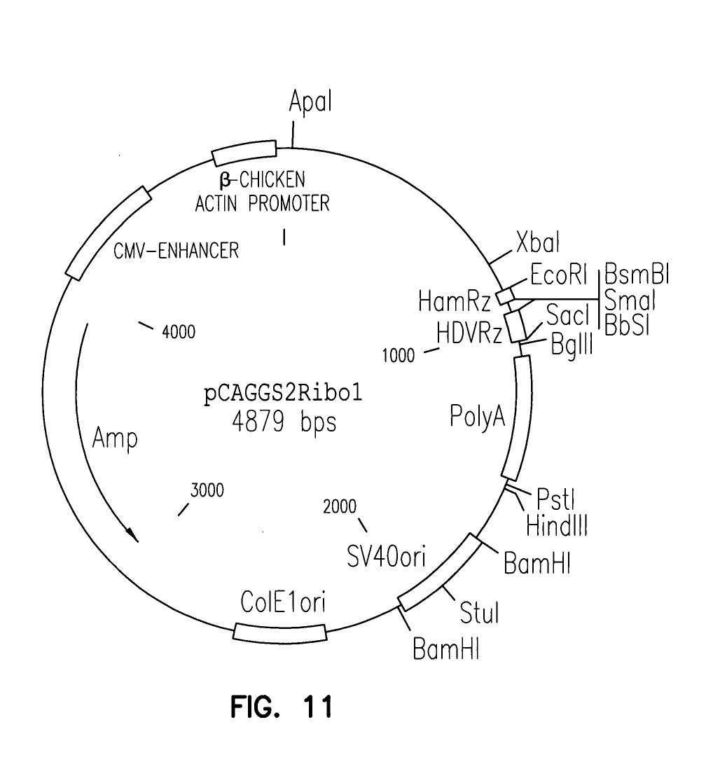

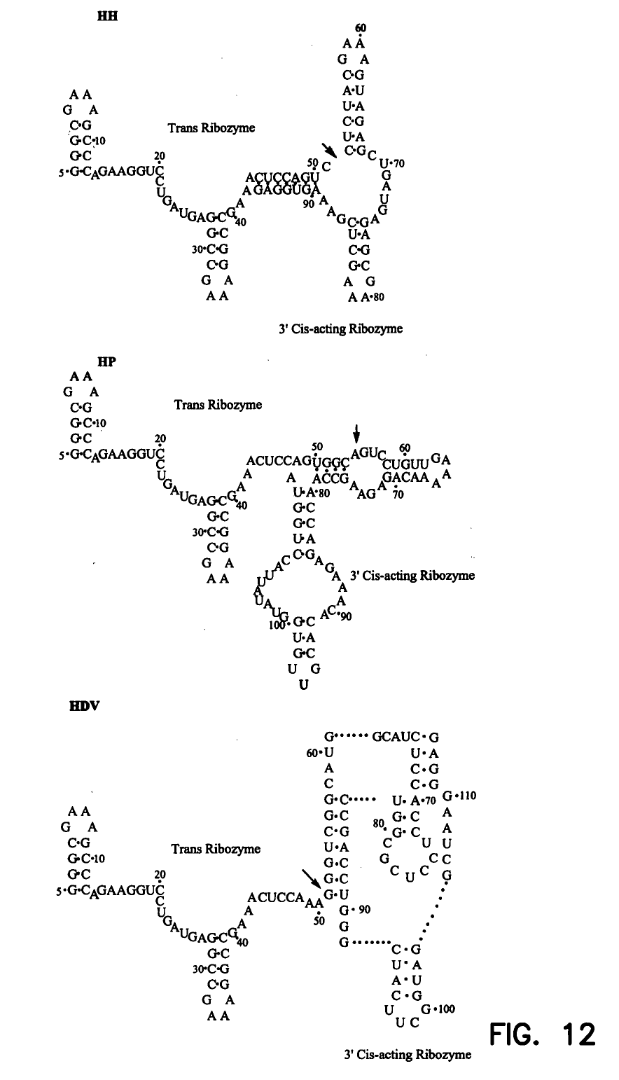

To produce vectors for a screening method, vectors were prepared that expressed influenza PA, PB1, PB2 and NP, and one for reporter vRNA production (FIGS. 8-9) or filovirus proteins and a reporter (FIG. 10). In one embodiment, the reporter luciferase is introduced between 3′ and 5′ NP noncoding sequences. The filovirus protein expression vectors, e.g., Marburg virus or Ebola virus vectors, include one or more of the following sequences: sequences for L, NP, VP30 and/or VP35. If more than one vector is employed, the vectors may be physically linked or each vector may be present on an individual plasmid or other, e.g., linear, nucleic acid delivery vehicle.

### REFERENCES

Albo et al., J. Virol., 70:9013 (1996).
Baron et al., J. Virol., 71:1265 (1997).
Bridgen et al., Proc. Natl. Acad. Sci. U.S.A., 93:15400 (1996).
Castrucci et al., J. Virol., 66:4647 (1992).
Castrucci et al., J. Virol., 69:2725 (1995).
Collins et al., Proc. Natl. Acad. Sci. U.S.A., 92:11563 (1995).
Dunn et al., Virology, 211:133 (1995).
Elliott, R. M. Mol. Med., 3:572 (1997).
Enami et al., Proc. Natl. Acad. Sci. U.S.A., 87:3802 (1990).
Enami et al., Virol., 65:2711 (1991).
Enami et al., Virology, 185:291 (1991).
Garcia-Sastre et al., Virus Res., 37:37 (1995).
Garcin et al., EMBO J., 14:6087 (1995).
Goto et al., Virology, 238:265 (1997).
Hagen et al., J. Virol., 68:1509 (1994).
He et al., Virology, 237:249 (1997).
Hoffman et al., J. Virol., 71:4272 (1997).
Honda et al., J. Biochem. (Tokyo), 104:1021 (1988).
Horimoto et al., J. Virol., 68:3120 (1994).
Huddleston et al., Nucl. Acids Res., 10:1029 (1982).
Jin et al., EMBO J., 13:5504 (1994).
Jin et al., EMBO J., 16:1236 (1997).
Kato et al., Genes Cells, 1:569 (1996).
Keitel, W. A. & Piedra, P. A. in Textbook of Influenza, eds. Nickolson, K. G., Webster, R. G., and Hay, A. (Blackwell, Oxford), pp. 373-390 (1998).
Kovesdi et al., Curr. Opin. Biotechnol., 8:583 (1997).
Lawson et al., Proc. Natl. Acad. Sci. U.S.A., 92:4477 (1995).
Leahy et al., J. Virol., 71:8347 (1997).
Leahy et al., J. Virol., 71:8352 (1997)
Leahy et al., J. Virol., 72:2305 (1998).
Li et al., Virus Res., 37:153 (1995).
Luytjes et al., Cell, 59:1107 (1989).
Mena et al., J. Virol., 70:5016 (1996).
Miyoshi et al., J. Virol., 72:8150 (1998).
Morita et al., Gene Ther., 7:1063 (2000).
Naim et al., J. Virol., 67:4831 (1993).
Neumann et al., J. Virol., 71:9690 (1997).
Neumann et al., J. Virol., 74:547 (2000).
Neumann et al., Proc. Natl. Acad. Sci. USA, 96:9345 (1999).
Neumann et al., Virology, 202:477 (1994).
Niwa et al., Gene, 108:193 (1991).
Onishi et al., Exp. Hematol., 24:324 (1996).
Palese et al., Proc. Natl. Acad. Sci. U.S.A., 93:11354 (1996).
Perez et al., Virology, 249:52 (1998).
Pleschka et al., J. Virol., 70:4188 (1996).
Radecke et al., EMBO J., 14:5773 (1995).
Schnell et al., EMBO J., 13:4195 (1994).
Subbarao et al., J. Virol., 67:7223 (1993).
Weber et al., Arch. Virol., 142:1029 (1997).
Weber et al., J. Virol., 70:8361 (1996).
Whelan et al., Proc. Natl. Acad. Sci. U.S.A., 92:8388 (1995).
Yasuda et al., J. Virol., 68:8141 (1994).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to identify modulators of influenza virus RNA polymerase, comprising:
a) providing a host cell comprising influenza virus vectors comprising a vector for protein expression comprising a promoter operably linked to an influenza virus PA DNA linked to a transcription termination sequence, a vector for protein expression comprising a promoter operably linked to an influenza virus PB1 DNA linked to a transcription termination sequence, a vector for protein expression comprising a promoter operably linked to an influenza virus PB2 DNA linked to a transcription termination sequence, a vector for protein expression comprising a promoter operably linked to an influenza virus NP DNA linked to a transcription termination sequence, and a vector for vRNA expression comprising a promoter operably linked to 5′ noncoding influenza virus sequences linked to reporter sequences linked to 3′ noncoding influenza virus sequences linked to a transcription termination sequence;
b) contacting the host cell and at least one agent; and
c) determining whether the one or more agents modulate the amount or activity of the one or more of the polymerase proteins.

2. The method of claim 1 wherein the vectors are each on a separate plasmid.

3. The method of claim 1 wherein the vectors with PA DNA, PB1 DNA and PB2 DNA are on the same plasmid.

4. The method of claim 1 wherein the promoter linked to the reporter sequences is a T7 RNA polymerase promoter.

5. The method of claim 1 wherein the reporter sequences are flanked by one or more ribozymes.

6. The method of claim 5 wherein the ribozyme is a tetrahymena ribozyme, RNase P, hammerhead ribozyme, hairpin ribozyme, hepatitis ribozyme, or synthetic ribozyme.

7. The method of claim 4 wherein the host cell further comprises T7 RNA polymerase.

8. The method of claim 1 wherein the promoter for vRNA expression comprises a RNA polymerase I promoter, RNA polymerase III promoter, T7 promoter, or T3 promoter.

9. The method of claim 1 wherein the promoter for protein expression comprises a RNA polymerase II promoter.

10. The method of claim 1 wherein each promoter for protein expression is the same.

11. The method of claim 1 wherein each promoter for protein expression is different.

12. The method of claim 1 wherein the transcription termination sequence of the vector for vRNA expression comprises a RNA polymerase I transcription termination sequence, RNA polymerase III transcription termination sequence, or a ribozyme.

13. The method of claim 1 wherein the reporter sequence is flanked by 5′ and 3′ noncoding sequences for influenza virus NP.

14. The method of claim 1 wherein the host cell does not produce infectious virus.

15. The method of claim 1 wherein at least one agent is siRNA.

16. The method of claim 1 wherein at least one agent is cDNA from a eukaryotic host cell.

17. The method of claim 1 wherein the reporter is a bioluminescent reporter.

18. The method of claim 17 wherein bioluminescence in the presence of the one or more agents is compared to bioluminescence in the absence of the agent(s).

19. The method of claim 1 wherein the activity in the presence of the one or more agents is compared to the activity in the absence of the agent(s).

20. The method of claim 1 wherein at least one agent modulates viral transcription.

21. The method of claim 1 wherein at least one agent inhibits viral transcription.

22. The method of claim 1 wherein the host cell is a mammalian host cell.

23. The method of claim 1 wherein the host cell is an avian, rodent, canine, feline, bovine, caprine, ovine, equine, swine, nonhuman primate or human cell.

24. An agent identified by the method of claim 1.
